(19) United States
(12) Patent Application Publication
(10) Pub. No.: US 2022/0375616 A1
(43) Pub. Date: Nov. 24, 2022

(54) METHOD, APPARATUS AND COMPUTER PROGRAM PRODUCT FOR GENERATING ACTIVITY SCORE DATA OBJECTS AND COMPOSITE SCORE DATA OBJECTS

(71) Applicant: Optum, Inc., Minnetonka, MN (US)

(72) Inventors: Lun YU, San Francisco, CA (US); Huzaifa SIAL, Emeryville, CA (US); Pooja RAO, Millbrae, CA (US)

(21) Appl. No.: 17/445,270

(22) Filed: Aug. 17, 2021

Related U.S. Application Data
(60) Provisional application No. 63/190,396, filed on May 19, 2021.

(51) Int. Cl. G16H 50/30; G16H 50/70; G06F 16/28

(52) U.S. Cl. CPC G16H 50/30; G16H 50/70; G06F 16/285; G16H 40/67

(57) ABSTRACT

Methods, apparatuses, systems, computing devices, computing entities, and/or the like are provided. An example method may include retrieving a client profile data object based at least in part on a client identifier indicator, determining a plurality of point indicators based at least in part on the at least one client activity data object, generating an activity score data object associated with the client profile data object, generating a composite score data object, and performing at least one score-based action based at least in part on the composite score data object and the client profile data object.

### CROSS-REFERENCE TO RELATED APPLICATIONS

This application claims priority to and the benefit of U.S. Provisional Application No. 63/190,396, titled “Personalized Index Score For Motivating Health Activities” and filed May 19, 2021, the entire content of which is incorporated herein by reference in its entirety.

### TECHNOLOGICAL FIELD

Embodiments of the present disclosure relate generally to improving computer and data system functionalities, such as, but not limited to, functionalities of data analytics systems. For example, various embodiments of the present disclosure may programmatically generate one or more composite score data objects based at least in part on one or more health score data objects and one or more activity score data objects and perform at least one score-based action based at least in part on the composite score data object.

### BACKGROUND

Data analytics systems have great potential for providing various technical advancement and technical benefits not only in the field of computer science, but also in other associated technical fields and applications. Applicant has identified many technical challenges, deficiencies and problems associated with data analytics systems and methods.

### BRIEF SUMMARY

In general, embodiments of the present disclosure provide methods, apparatuses, systems, computing devices, computing entities, and/or the like.

In accordance with various embodiments of the present disclosure, an apparatus is provided. The apparatus may comprise at least one processor and at least one non-transitory memory comprising a computer program code. The at least one non-transitory memory and the computer program code may be configured to, with the at least one processor, cause the apparatus to retrieve a client profile data object based at least in part on a client identifier indicator, wherein the client profile data object is associated with a health score data object and at least one client activity data object; determine a plurality of point indicators based at least in part on the at least one client activity data object, wherein the plurality of point indicators are associated with a plurality of category indicators and a plurality of type indicators; generate, based at least in part on the plurality of category indicators, the plurality of type indicators, the plurality of point indicators, and the health score data object, an activity score data object associated with the client profile data object; generate a composite score data object based at least in part on the health score data object and the activity score data object, wherein the composite score data object is associated with the client profile data object; and perform at least one score-based action based at least in part on the composite score data object and the client profile data object.

In some embodiments, when determining the plurality of point indicators, the at least one non-transitory memory and the computer program code are configured to, with the at least one processor, cause the apparatus to: determine a point indicator of the plurality of point indicators based at least in part on a category indicator of the plurality of category indicators, wherein the point indicator is associated with the category indicator.

In some embodiments, when determining the plurality of point indicators, the at least one non-transitory memory and the computer program code are configured to, with the at least one processor, cause the apparatus to: determine a point indicator of the plurality of point indicators based at least in part on a type indicator of the plurality of type indicators, wherein the point indicator is associated with the type indicator.

In some embodiments, when determining the plurality of point indicators, the at least one non-transitory memory and the computer program code are configured to, with the at least one processor, cause the apparatus to: determine a point indicator of the plurality of point indicators based at least in part on a category indicator of the plurality of category indicators and a type indicator of the plurality of type indicators, wherein the point indicator is associated with the category indicator and the type indicator.

In some embodiments, the health score data object comprises a health category score indicator, wherein the health category score indicator is associated with a category indicator of the plurality of category indicators.

In some embodiments, when generating the activity score data object, the at least one non-transitory memory and the computer program code are configured to, with the at least one processor, cause the apparatus to: generate an activity category score indicator associated with the category indicator based at least in part on the health category score indicator.

In some embodiments, when generating the activity category score indicator, the at least one non-transitory memory and the computer program code are configured to, with the at least one processor, cause the apparatus to: determine a gap indicator based at least in part on the health category score indicator and a health category maximum indicator associated with the category indicator; and determine a decay indicator based at least in part on the gap indicator and at least one adjustable indicator.

In some embodiments, a point indicator of the plurality of point indicators is associated with the category indicator and a type indicator of the plurality of type indicators. In some embodiments, the at least one non-transitory memory and the computer program code are configured to, with the at least one processor, cause the apparatus to: determine a type denominator indicator associated with the type indicator based at least in part on the point indicator, a maximum activity indicator associated with the type indicator, and the decay indicator; and determine a type numerator indicator associated with the type indicator based at least in part on the point indicator, an actual activity indicator associated with the type indicator and the client profile data object, and the decay indicator.

In some embodiments, the at least one non-transitory memory and the computer program code are configured to, with the at least one processor, cause the apparatus to: generate an activity type score indicator based at least in part on the type denominator indicator and the type numerator indicator, wherein the activity type score indicator is associated with the type indicator and the category indicator.

In some embodiments, when generating the activity category score indicator, the at least one non-transitory memory and the computer program code are configured to, with the at least one processor, cause the apparatus to: generate a plurality of type denominator indicators and a plurality of type numerator indicators associated with the category indicator; and aggregate at least the plurality of type denominator indicators and the plurality of type numerator indicators to generate the activity category score indicator.

In some embodiments, when generating the composite score data object, the at least one non-transitory memory and the computer program code are configured to, with the at least one processor, cause the apparatus to: generate a plurality of activity category score indicators associated with the plurality of category indicators, wherein the plurality of activity category score indicators comprises a plurality of category denominator indicators and a plurality of category numerator indicators; determine a plurality of gap indicators associated with the plurality of category indicators based at least in part on a plurality of health category score indicators associated with the health score data object; and generate the activity score data object based at least in part on the plurality of category denominator indicators, the plurality of category numerator indicators, and the plurality of gap indicators.

In some embodiments, the at least one non-transitory memory and the computer program code are configured to, with the at least one processor, cause the apparatus to: receive an activity update data object associated with the client identifier indicator, wherein the activity update data object comprises an updated actual activity indicator associated with a category indicator and a type indicator; in response to receiving the activity update data object: generate an updated type numerator indicator and an updated type denominator indicator associated with the type indicator based at least in part on the updated actual activity indicator; generate an updated activity category score indicator associated with the category indicator based at least in part on the updated type numerator indicator and the updated type denominator indicator; generate an updated activity score data object based at least in part on the updated activity category score indicator; and generate an updated composite score data object based at least in part on the updated activity score data object.

In some embodiments, the composite score data object is associated with a composite score data object creation time indicator, wherein the at least one non-transitory memory and the computer program code are configured to, with the at least one processor, cause the apparatus to: determine an activity update time interval indicator; determine whether a time period between the composite score data object creation time indicator and a current time indicator satisfies the activity update time interval indicator; and in response to determining that the time period satisfies the activity update time interval indicator: determine an updated actual activity indicator associated with at least one of a category indicator or a type indicator; generate an updated type numerator indicator and an updated type denominator indicator associated with the type indicator based at least in part on the updated actual activity indicator; generate an updated activity category score indicator associated with the category indicator based at least in part on the updated type numerator indicator and the updated type denominator indicator; generate an updated activity score data object based at least in part on the updated activity category score indicator; and generate an updated composite score data object based at least in part on the updated activity score data object.

In some embodiments, when performing the at least one score-based action, the at least one non-transitory memory and the computer program code are configured to, with the at least one processor, cause the apparatus to: render, on a user interface displayed on a client computing entity associated with the client identifier indicator, the composite score data object.

In some embodiments, when performing the at least one score-based action, the at least one non-transitory memory and the computer program code are configured to, with the at least one processor, cause the apparatus to: generate at least one activity recommendation data object based at least in part on the composite score data object and the client profile data object; and render, on a user interface displayed on a client computing entity associated with the client identifier indicator, the at least one activity recommendation data object.

In some embodiments, the client profile data object comprises a client preference indicator, wherein the at least one non-transitory memory and the computer program code are configured to, with the at least one processor, cause the apparatus to: generate the at least one activity recommendation data object based further on the client preference indicator.

In accordance with various embodiments of the present disclosure, a computer-implemented method is provided. The computer-implemented method may comprise retrieving a client profile data object based at least in part on a client identifier indicator, wherein the client profile data object is associated with a health score data object and at least one client activity data object; determining a plurality of point indicators based at least in part on the at least one client activity data object, wherein the plurality of point indicators are associated with a plurality of category indicators and a plurality of type indicators; generating, based at least in part on the plurality of category indicators, the plurality of type indicators, the plurality of point indicators, and the health score data object, an activity score data object associated with the client profile data object; generating a composite score data object based at least in part on the health score data object and the activity score data object, wherein the composite score data object is associated with the client profile data object; and performing at least one score-based action based at least in part on the composite score data object and the client profile data object.

In accordance with various embodiments of the present disclosure, a computer program product is provided. The computer program product may comprise at least one non-transitory computer-readable storage medium having computer-readable program code portions stored therein. The computer-readable program code portions may comprise an executable portion configured to retrieve a client profile data object based at least in part on a client identifier indicator, wherein the client profile data object is associated with a health score data object and at least one client activity data object; determine a plurality of point indicators based at least in part on the at least one client activity data object, wherein the plurality of point indicators are associated with a plurality of category indicators and a plurality of type indicators; generate, based at least in part on the plurality of category indicators, the plurality of type indicators, the plurality of point indicators, and the health score data object, an activity score data object associated with the client profile data object; generate a composite score data object based at least in part on the health score data object and the activity score data object, wherein the composite score data object is associated with the client profile data object; and perform at least one score-based action based at least in part on the composite score data object and the client profile data object.

The above summary is provided merely for purposes of summarizing some example embodiments to provide a basic understanding of some aspects of the disclosure. Accordingly, it will be appreciated that the above-described embodiments are merely examples. It will be appreciated that the scope of the disclosure encompasses many potential embodiments in addition to those here summarized, some of which will be further described below.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 is a diagram of an example activity score data object and/or composite score data object generating platform/system that can be used in accordance with various embodiments of the present disclosure;

FIG. 2 is a schematic representation of an example data object computing entity in accordance with various embodiments of the present disclosure;

FIG. 3 is a schematic representation of an example client computing entity in accordance with various embodiments of the present disclosure;

FIGS. 4, 5, 6, 7, 8, 9, 10, 11, 12, 13A, 13B, 14, and 15 provide example flowcharts and diagrams illustrating example steps, processes, procedures, and/or operations associated with an example activity score data object and/or composite score data object generating platform/system in accordance with various embodiments of the present disclosure; and

FIG. 16 provides an example view of an example user interface associated with an example activity score data object and/or composite score data object generating platform/system in accordance with various embodiments of the present disclosure.

### DETAILED DESCRIPTION OF SOME EXAMPLE EMBODIMENTS

Various embodiments of the present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. The term “or” (also designated as “/”) is used herein in both the alternative and conjunctive sense, unless otherwise indicated. The terms “illustrative” and “exemplary” are used to be examples with no indication of quality level. Like numbers refer to like elements throughout. The phrases “in one embodiment,” “according to one embodiment,” and/or the like generally mean that the particular feature, structure, or characteristic following the phrase may be included in at least one embodiment of the present disclosure and may be included in more than one embodiment of the present disclosure (importantly, such phrases do not necessarily refer to the same embodiment).

### I. COMPUTER PROGRAM PRODUCTS, METHODS, AND COMPUTING ENTITIES

Embodiments of the present disclosure may be implemented as computer program products that comprise articles of manufacture. Such computer program products may include one or more software components including, for example, applications, software objects, methods, data structures, and/or the like. A software component may be coded in any of a variety of programming languages. An illustrative programming language may be a lower-level programming language such as an assembly language associated with a particular hardware architecture and/or operating system platform/system. A software component comprising assembly language instructions may require conversion into executable machine code by an assembler prior to execution by the hardware architecture and/or platform/system. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Additionally, or alternatively, embodiments of the present disclosure may be implemented as a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media may include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatuses, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### II. EXEMPLARY SYSTEM ARCHITECTURE

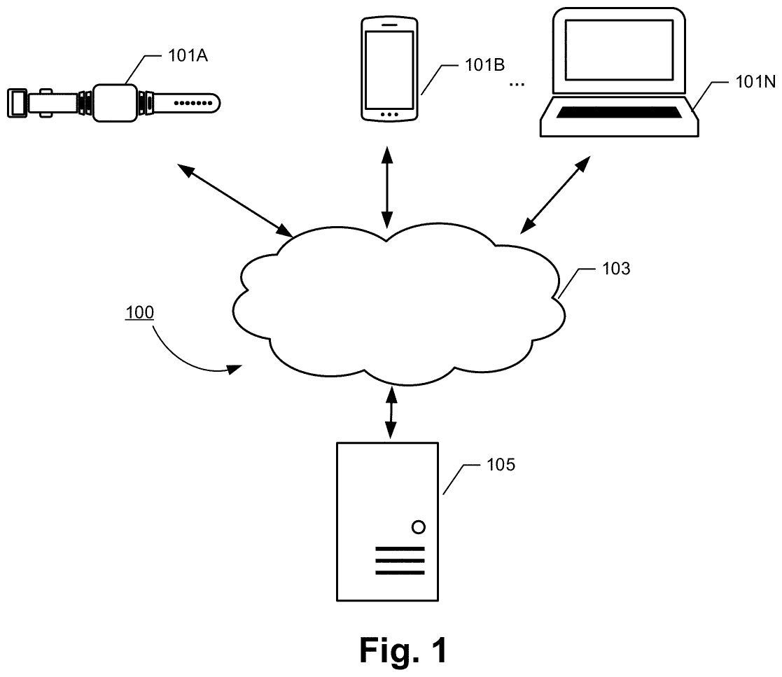

FIG. 1 provides an illustration of an activity score data object and/or composite score data object generating platform/system 100 that can be used in conjunction with various embodiments of the present disclosure. As shown in FIG. 1, the activity score data object and/or composite score data object generating platform/system 100 may comprise one or more data object computing entities 105, one or more client computing entities 101A, 101B . . . 101N, and one or more networks 103. Each of the components of the activity score data object and/or composite score data object generating platform/system 100 may be in electronic communication with, for example, one another over the same or different wireless or wired networks 103 including, for example, a wired or wireless Personal Area Network (PAN), Local Area Network (LAN), Metropolitan Area Network (MAN), Wide Area Network (WAN), and/or the like. Additionally, while FIG. 1 illustrates certain system entities as separate, standalone entities, the various embodiments are not limited to this particular architecture.

a. Exemplary Data Object Computing Entity

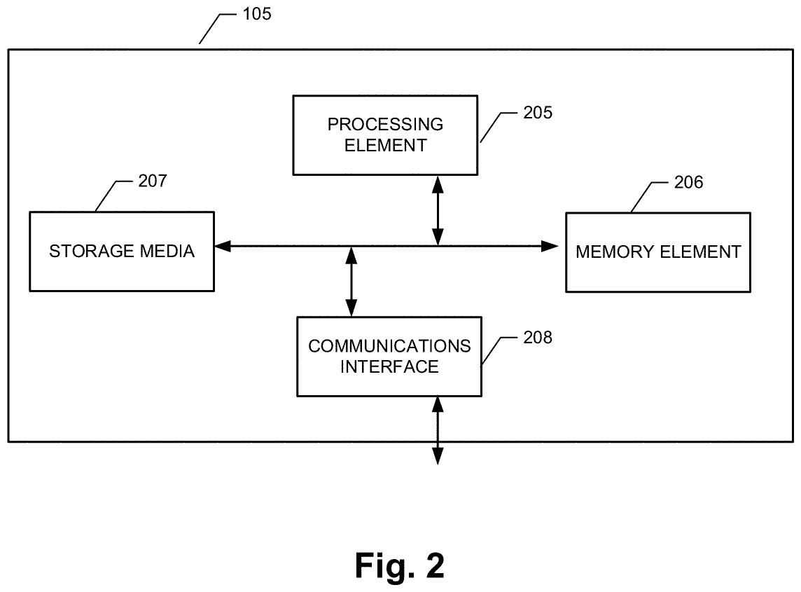

FIG. 2 provides a schematic of a data object computing entity 105 according to one embodiment of the present disclosure. In general, the terms computing entity, entity, device, system, and/or similar words used herein interchangeably may refer to, for example, one or more computers, computing entities, desktop computers, mobile phones, tablets, phablets, notebooks, laptops, distributed systems, items/devices, terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein. In one embodiment, these functions, operations, and/or processes can be performed on data, content, information, and/or similar terms used herein.

As indicated, in one embodiment, the data object computing entity 105 may also include one or more network and/or communications interface 208 for communicating with various computing entities, such as by communicating data, content, information, and/or similar terms used herein that can be transmitted, received, operated on, processed, displayed, stored, and/or the like. For instance, the data object computing entity 105 may communicate with other data object computing entities 105, one or more client computing entities 101A-101N, and/or the like.

As shown in FIG. 2, in one embodiment, the data object computing entity 105 may include or be in communication with one or more processing elements (for example, processing element 205) (also referred to as processors, processing circuitry, and/or similar terms used herein interchangeably) that communicate with other elements within the data object computing entity 105 via a bus, for example, or network connection. As will be understood, the processing element 205 may be embodied in a number of different ways. For example, the processing element 205 may be embodied as one or more complex programmable logic devices (CPLDs), microprocessors, multi-core processors, coprocessing entities, application-specific instruction-set processors (ASIPs), and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

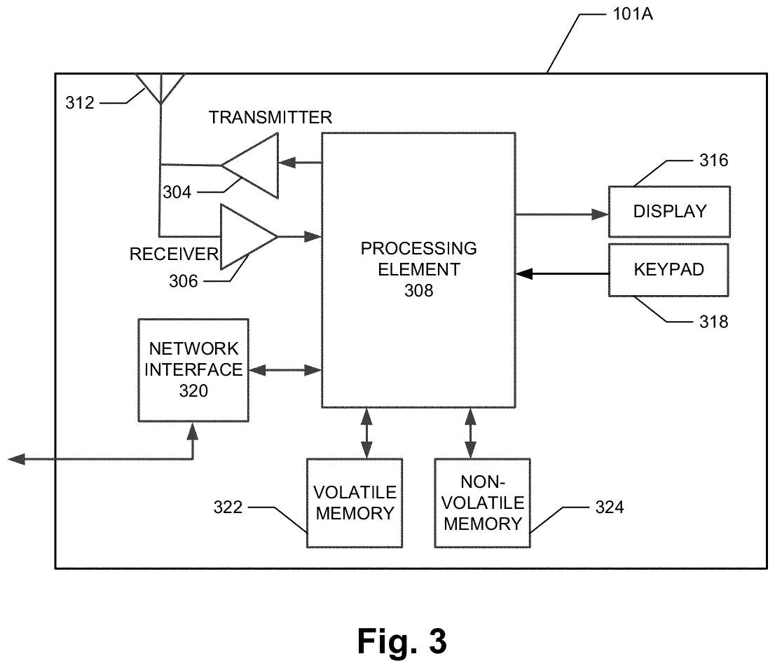

In one embodiment, the data object computing entity 105 may further include or be in communication with volatile media (also referred to as volatile storage, memory, memory storage, memory circuitry and/or similar terms used herein interchangeably). In one embodiment, the volatile storage or memory may also include one or more memory element 206 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory element 206 may be used to store at least portions of the databases, database instances, database management system entities, data, applications, programs, program modules, scripts, source code, object code, byte code, compiled code, interpreted code, machine code, executable instructions, and/or the like being executed by, for example, the processing element 205 as shown in FIG. 2 and/or the processing element 308 as described in connection with FIG. 3. Thus, the databases, database instances, database management system entities, data, applications, programs, program modules, scripts, source code, object code, byte code, compiled code, interpreted code, machine code, executable instructions, and/or the like may be used to control certain aspects of the operation of the data object computing entity 105 with the assistance of the processing element 205 and operating system.

In one embodiment, the data object computing entity 105 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or storage media 207 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or storage media 207 may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to refer to a structured or unstructured collection of information/data that is stored in a computer-readable storage medium.

Storage media 207 may also be embodied as a data storage device or devices, as a separate database server or servers, or as a combination of data storage devices and separate database servers. Further, in some embodiments, storage media 207 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. An example of the embodiments contemplated herein would include a cloud data storage system maintained by a third-party provider and where some or all of the information/data required for the operation of the recovery prediction system may be stored. Further, the information/data required for the operation of the recovery prediction system may also be partially stored in the cloud data storage system and partially stored in a locally maintained data storage system. More specifically, storage media 207 may encompass one or more data stores configured to store information/data usable in certain embodiments.

As indicated, in one embodiment, the data object computing entity 105 may also include one or more network and/or communications interface 208 for communicating with various computing entities, such as by communicating data, content, information, and/or similar terms used herein interchangeably that can be transmitted, received, operated on, processed, displayed, stored, and/or the like. For instance, the data object computing entity 105 may communicate with computing entities or communication interfaces of other data object computing entities 105, client computing entities 101A-101N, and/or the like.

As indicated, in one embodiment, the data object computing entity 105 may also include one or more network and/or communications interface 208 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the data object computing entity 105 may be configured to communicate via wireless external communication networks using any of a variety of protocols, such as general packet radio service (GPRS), Universal Mobile Telecommunications System (UMTS), Code Division Multiple Access 1900 (CDMA1900), CDMA1900 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), Institute of Electrical and Electronics Engineers (IEEE) 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The data object computing entity 105 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the data object computing entity's components may be located remotely from components of other data object computing entities 105, such as in a distributed system. Furthermore, one or more of the components may be aggregated and additional components performing functions described herein may be included in the data object computing entity 105. Thus, the data object computing entity 105 can be adapted to accommodate a variety of needs and circumstances.

b. Exemplary Client Computing Entity

FIG. 3 provides an illustrative schematic representative of one of the client computing entities 101A to 101N that can be used in conjunction with embodiments of the present disclosure. As will be recognized, the client computing entity may be operated by an agent and include components and features similar to those described in conjunction with the data object computing entity 105. Further, as shown in FIG. 3, the client computing entity may include additional components and features. For example, the client computing entity 101A can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various entities, such as a data object computing entity 105, another client computing entity 101A, and/or the like. In this regard, the client computing entity 101A may be capable of operating with one or more air interface standards, communication protocols, modulation types, and access types. More particularly, the client computing entity 101A may comprise a network interface 320, and may operate in accordance with any of a number of wireless communication standards and protocols. In a particular embodiment, the client computing entity 101A may operate in accordance with multiple wireless communication standards and protocols, such as GPRS, UMTS, CDMA1900, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the client computing entity 101A can communicate with various other entities using Unstructured Supplementary Service data (USSD), Short Message Service (SMS), Multimedia Messaging Service (MMS), Dual-Tone Multi-Frequency (DTMF) Signaling, Subscriber Identity Module Dialer (SIM dialer), and/or the like. The client computing entity 101A can also download changes, add-ons, and updates, for instance, to its firmware, software (e.g., including executable instructions, applications, program modules), and operating system.

According to one embodiment, the client computing entity 101A may include location determining aspects, devices, modules, functionalities, and/or similar words used herein interchangeably. For example, the client computing entity 101A may include outdoor positioning aspects, such as a location module adapted to acquire, for example, latitude, longitude, altitude, geocode, course, direction, heading, speed, UTC, date, and/or various other information/data. In one embodiment, the location module can acquire data, sometimes known as ephemeris data, by identifying the number of satellites in view and the relative positions of those satellites. The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. Alternatively, the location information/data/data may be determined by triangulating the position in connection with a variety of other systems, including cellular towers, Wi-Fi access points, and/or the like. Similarly, the client computing entity 101A may include indoor positioning aspects, such as a location module adapted to acquire, for example, latitude, longitude, altitude, geocode, course, direction, heading, speed, time, date, and/or various other information/data. Some of the indoor aspects may use various position or location technologies including Radio-Frequency Identification (RFID) tags, indoor beacons or transmitters, Wi-Fi access points, cellular towers, nearby computing devices (e.g., smartphones, laptops) and/or the like. For instance, such technologies may include iBeacons, Gimbal proximity beacons, Bluetooth Low Energy (BLE) transmitters, Near Field Communication (NFC) transmitters, and/or the like. These indoor positioning aspects can be used in a variety of settings to determine the location of someone or something to within inches or centimeters.

The client computing entity 101A may also comprise a user interface comprising one or more user input/output interfaces (e.g., a display 316 and/or speaker/speaker driver coupled to a processing element 308 and a touch screen, keyboard, mouse, and/or microphone coupled to a processing element 308). For example, the user output interface may be configured to provide an application, browser, user interface, dashboard, webpage, and/or similar words used herein interchangeably executing on and/or accessible via the client computing entity 101A to cause display or audible presentation of information/data and for user interaction therewith via one or more user input interfaces. The user output interface may be updated dynamically from communication with the data object computing entity 105. The user input interface can comprise any of a number of devices allowing the client computing entity 101A to receive data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the client computing entity 101A and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the client computing entity 101A can collect information/data, user interaction/input, and/or the like.

The client computing entity 101A can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, data, applications, programs, program modules, scripts, source code, object code, byte code, compiled code, interpreted code, machine code, executable instructions, and/or the like to implement the functions of the client computing entities 101A-101N.

c. Exemplary Networks

In one embodiment, the networks 103 may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks 103 may have any suitable communication range associated therewith and may include, for example, global networks (e.g., the Internet), MANs, WANs, LANs, or PANs. In addition, the networks 103 may include medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms/systems provided by network providers or other entities.

Further, the networks 103 may utilize a variety of networking protocols including, but not limited to, TCP/IP based networking protocols. In some embodiments, the protocol is a custom protocol of JavaScript Object Notation (JSON) objects sent via a WebSocket channel. In some embodiments, the protocol is JSON over RPC, JSON over REST/HTTP, and/or the like.

### III. EXEMPLARY OPERATION

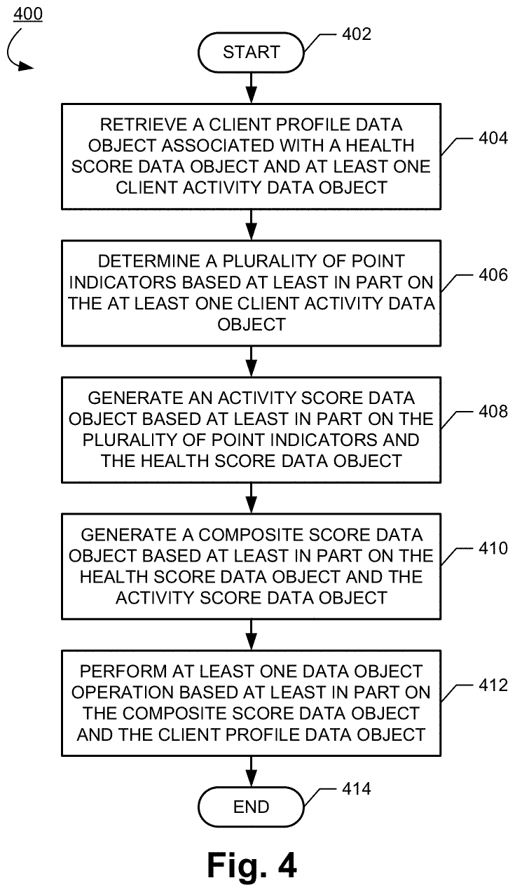

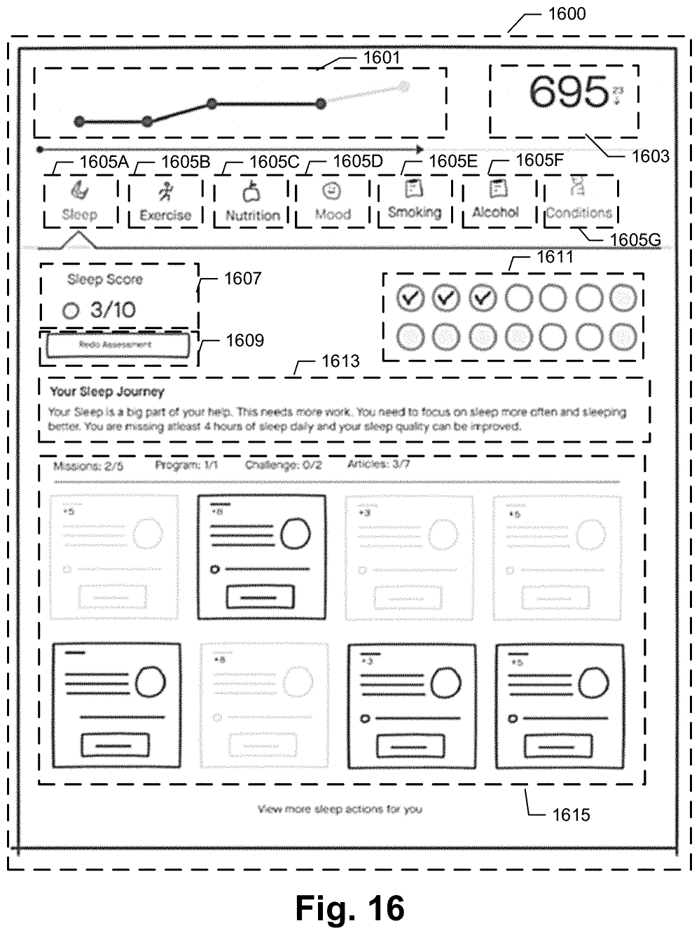

Reference will now be made to FIGS. 4, 5, 6, 7, 8, 9, 10, 11, 12, 13A, 13B, 14, 15, and 16. In particular, FIGS. 4, 5, 6, 7, 8, 9, 10, 11, 12, 13A, 13B, 14, and 15 provide flowcharts and diagrams illustrating example steps, processes, procedures, and/or operations associated with an activity score data object and/or composite score data object generating platform/system and/or a data object computing entity in accordance with various embodiments of the present disclosure. FIG. 16 provides an example view of an example user interface associated with an example activity score data object and/or composite score data object generating platform/system and/or a client computing entity in accordance with various embodiments of the present disclosure

While example embodiments of the present disclosure may be described in the context of healthcare, as will be recognized, embodiments of the present disclosure are not limited to this context only.

### a. Overview

A “data analytics system” may refer to a type of system that utilizes computing resources that include, but not limited to, computing resources in hardware forms (such as, but not limited to, computers, servers, and/or the like) and/or computing resources in software forms (such as, but not limited to, software applications, firmware, and/or the like) to conduct data analysis operations (such as, but not limited to, analyzing raw data and/or generating new data based at least in part on the analysis of the raw data). Data analytics system may be implemented or applied in a variety of scenarios and situations.

For example, an example data analytics system may generate a summary or overview of raw data that represents an overall picture of the raw data. As an example, an example data analytics system may be implemented or applied to analyze raw data to generate a health metric that provides a summary or overview of a personal health level associated with a user (for example, representing an overall picture of how healthy the user is). As another example, an example data analytics system may be implemented or applied to analyze raw data to generate an activity metric that provides a summary or overview of a personal activity level associated with a user (for example, representing an overall picture of how active the user is).

Additionally, or alternatively, an example data analytics system may examine raw data and infer one or more trends based at least in part on the raw data. As an example, an example data analytics system may be implemented or applied to analyze raw data to infer one or more trends associated with a user's personal health based at least in part on the analysis of raw data. As another example, an example data analytics system may be implemented or applied to analyze raw data to infer one or more trends associated with a user's personal activity based at least in part on the analysis of raw data.

Additionally, or alternatively, an example data analytics system may generate recommendations based at least in part on the analysis of the raw data. Continuing from the example above, an example data analytics system may be implemented or applied to analyze raw data that represents or is associated with a user's activity information, and/or generate recommendations for the user's activity based at least in part on the analysis of raw data. To motivate users to develop good health behaviors and engage in healthy activities, an example data analytics system may implement a reward mechanism. For example, an example data analytics system may provide reward points to a user when the user completes health activities and programs.

As described above, an example data analytics system conducts its data analysis operations based at least in part on raw data. In some examples, raw data may be generated by the data analytics system and/or by a data collection system that stores such raw data in a database. A “data collection system” may refer to a type of system that utilizes computing resources including, but not limited to, computing resources in hardware forms (such as, but not limited to, computers, servers, and/o the like) and/or computing resources in software forms (such as, but not limited to, software applications, firmware, and/or the like) to record, collect, gather, and/or track data and/or information and/or to generate raw data based at least in part on the data and/or information that has been recorded, collected, gathered, and/or tracked.

Continuing from the health/activity example above, raw data that represents or is associated with a user's health information or activity information may be generated by the data analytics system and/or by a data collection system that stores such raw data in a database. An example data collection system in this example may include computing resources in hardware forms and/or software forms. For example, the example data collection system may include computing resources such as, but not limited to, a wearable computer (such as, but not limited to, fitness trackers, smart watches, and/or the like) and/or a mobile computer (such as, but not limited to, smartphones, tablet computers, and/or the like) that monitor and/or track health and/or fitness related information associated with a user. Additionally, or alternatively, the example data collection system may include computing resources such as, but not limited to, software applications that run on a wearable computer and/or a mobile computer, and such software applications enable a user to record or input data and/or information related to his or her health and/or fitness. As such, the example data collection system may generate raw data that represents health and/or fitness information associated with a user, and may store the raw data in a database. In some examples, a data analytics system may retrieve such raw data from the database to conduct data analysis operations as described above.

However, many data analytics systems are faced with many technical challenges and plagued by many technical limitations in conducting data analytics operations, and such technical challenges and technical limitations adversely affect computer and data system functionalities.

For example, a data collection system may record, collect, gather, and/or track a large amount of information and generate a large amount of raw data. Such raw data may be constructed or stored in a distributed manner. As a result, data correlations between and among different parts of the raw data cannot be easily identified. When a data analytics system analyzes the raw data to generate a summary or overview that represents the raw data, the data analytics system may not identify all the relevant raw data, and therefore may generate a summary or overview that is distorted from what the raw data actually represents. When the data analytics system analyzes the raw data to infer one or more trends, the data analytics system may not identify all the relevant raw data, and therefore may infer trends that are not accurate reflections of what the raw data actually represents. When the data analytics system analyzes the raw data to generate recommendations, the data analytics system may not identify all the relevant raw data, and therefore may generate recommendations that are not applicable or lack utility.

Continuing from the health/activity example above, many data analytics systems may fail to identify the correlation between a user's health data and a user's activity data. User experience of these data analytics systems is fragmented because such data analytics systems do not take into account user's health data when analyzing user's activity data. Some data analytics systems may provide rewards to a user on activities that are not proportionate to the impacts on the user's health areas that need attention or improvement from the user. As an example, a user may have already developed a healthy sleeping habit, but does not have a healthy eating habit. In this example, the health area associated with diet needs more attention and improvements than the health area associated with sleep for this user. However, many data analytics systems may reward this user the same amount of reward points when the user engages in healthy sleeping activities as when the user engages in healthy eating activities. In other words, such systems fail to provide a health factor drive solution to motivate users to improve their health. Additionally, user experience of such data analytics systems suffers due to the failure to correlate a user's health data with a user's activity data in conducting data analysis operations, as activities that a user has taken have no effect on their health metric.

As another example, many data analytics systems fail to individualize or isolate raw data that is associated with a particular user in conducting data analysis operations. When a data analytics system analyzes the raw data to generate a summary or overview of a user, the data analytics system may use raw data associated with other users as basis for analysis, and therefore may generate a summary or overview that is distorted from what the raw data actually represents for that user. When the data analytics system analyzes the raw data to infer one or more trends associated with a user, the data analytics system uses raw data associated with other users as basis for identification, and therefore may infer trends that do not reflect any actual trend associated with the user. When the data analytics system analyzes the raw data to generate recommendations, the data analytics system may use raw data associated with other users as basis for recommendations, and therefore may generate recommendations that are not applicable to the user.

Continuing from the health/activity example above, many data analytics systems may allow users to earn rewards by accomplishing some health activities. However, those data analytics systems are not personalized at the user level, as such data analytics systems are based at least in part on population health risk rather than individual health needs. For example, some data analytics systems may generate a “daily wellness meter” that assigns a certain amount of points to each activity, and such amount of points is the same for all users. In this example, the same amount of points may be awarded to a physically inactive user for taking 1000 steps and to a physically active user for taking 1000 steps, despite the 1000 steps may provide more improvements on the health level of the physically inactive user than that of the physically active user. User experience suffers as these data analytics systems select standards and judge everyone based at least in part on the same standards while two persons are exactly alike, and a user may not know how the data analytics systems generate such metrics or how to change the metrics.

As another example, many data analytics systems do not provide any indication to end users that would explain or describe data, information, or factor affecting the summary or overview of raw data generated by the data analytics systems, and do not provide any indication to end users that would explain or describe how the recommendations are generated by the data analytics systems.

Continuing from the health/activity example above, many data analytics systems do not provide any explanation to users on how each activity can improve the health metrics associated with the user, and do not provide any explanation to the users as to which activity can improve which health metrics. Many data analytics systems do not give users a clear indication of changes in the metrics and potential actions that need to be taken to improve the metrics.

In contrast, various embodiments of the present disclosure overcome at least the above-referenced technical challenges and technical difficulties, provide various technical benefits, and improve computer and data system functionalities, such as, but not limited to, functionalities of data analytics systems.

For example, various embodiments of the present disclosure programmatically generate one or more composite score data objects based at least in part on one or more health score data objects and one or more activity score data objects, and perform at least one score-based action based at least in part on the composite score data object to overcome such technical challenges and technical difficulties, provide various technical benefits, and improve computer and data system functionalities.

Continuing from the health/activity example above, when generating the one or more composite score data objects, various embodiments of the present disclosure identify data correlations between and among health data and activity data, individualize and isolate health data and activity data that is associated with a particular user, and provide indication to end users that would explain how the recommendations on activities are generated.

For example, various embodiments of the present disclosure provide methods, systems, and apparatus that automatically update an activity score data object by tracking users' engagement in health activities and programs that tie to health benefits and based at least in part on the user's health status. The activity score data object comprises an activity score indicator that increases as users take more health activities and decreases as users disengage from health activities. Various embodiments of the present disclosure calculate the activity score indicator by factoring the user's health status and making reward points proportionate to the impact on user's health.

Additionally, various embodiments of the present disclosure provide methods, systems, and apparatus that generate a composite score data object factoring users' health status and user attributes. The composite score data object includes a composite score indicator, and users with underperforming health will be set a goal of accomplishing more relevant health activities and programs to improve the composite score indicator. When generating an activity recommendation data object, various embodiments of the present disclosure take into account user preferences, demographics, age, chronic condition, social determinants of health, etc., as well as changing burden of actions per user. As such, various embodiments of the present disclosure track users' health status, personalize the engagement experience for users based at least in part on their benefits, interests and abilities, and provide a framework that will scale with the user's health and preference.

Additionally, various embodiments of the present disclosure provide methods, systems, and apparatus that make activity score data objects and composite score data objects explainable to end users by aligning health activities and programs with the semantic health categories that can be expanded or removed depending on the individual user. For example, various embodiments of the present disclosure provide an interpretable interface for users to understand how to improve their health by engaging in what activities. Various embodiments of the present disclosure provide guidance by providing points for the impact of different activities based at least in part on the user's health while also providing instant gratification by changing the score upon completion of said activities.

As such, various embodiments of the present disclosure improve data analytics system functionalities and provide technical benefits on computer systems while improving health and well-being of end users, details of which are described herein.

### b. Exemplary Technical Advantages

Various embodiments of the present invention introduce techniques for generating activity scores that are configured to be used to recommend activities to an end-user of a device. For example, various embodiments of the present invention introduce techniques for generating activity scores based on point indicators associated with an activity, where each point indicator may be associated with a combination of an activity category indicator and an activity type indicator. By utilizing the noted techniques, various embodiments of the present invention enable techniques for expedited and relatively non-resource-intensive generation of activity scores that enable generating such scores on end-user client devices, rather than on server systems. This in turn reduces the need for client-server communications in order to generate activity scores, which in turn improves network efficiency and network reliability of distributed user activity monitoring systems.

For example, in some embodiments, the above-noted techniques can reduce the amount of network resources and/or the amount of network bandwidth needed to transmit client-server communications to generate an activity score using a server device that is remote from an end-user client device. Examples of such client-server communications requests for activity score generation, requests for updating activity scores, requests for data needed to generate activity scores, communications describing data related to result of activity score generation computations, and/or the like. As another example, in some embodiments, the above-noted techniques can reduce the number of client-server communications needed to generate an activity score using a server device that is remote from an end-user client device, thus lessening the probability that a distributed user activity monitoring system may face network failure.

### c. Definitions

In the present disclosure, the term “data object” may refer to a data structure that comprises, represents, indicates, and/or is associated with one or more attributes, functionalities and/or characteristics associated with data and/or information in an example activity score data object and/or composite score data object generating platform/system.

In the present disclosure, examples of data objects may be associated with a variety of types, including, but not limited to, client profile data objects, client activity data objects, health score data objects, activity score data objects, activity recommendation data object, activity update data object, composite score data object, details of which are described herein.

In the present disclosure, the term “indicator” may refer to a data value, a data field, a data unit or a data element that is received by, retrieved by, generated by, calculated by, determined by, and/or stored by an example activity score data object and/or composite score data object generating platform/system. In some examples, an example data object may be associated with one or more indicators. In such examples, the one or more indicators comprise, represent, describe, indicate, and/or are associated with one or more attributes, functionalities and/or characteristics associated with the example data object. In some embodiments, an example indicator may be associated with one or more other indicators.

In some embodiments, an example indicator may comprise one or more alphabetical characters, one or more numerical characters, one or more alphanumeric characters, one or more American Standard Code for Information Interchange (ASCII) texts, one or more pointers, one or more memory addresses, and/or the like.

In the present disclosure, examples of data objects may be associated with a variety of types, including, but not limited to, client identifier indicator, health score indicator, category indicator, health category score indicator, health category maximum indicator, gap indicator, decay indicator, type indicator, point indicator, activity type score indicator, type denominator indicator, type numerator indicator, actual activity indicator, maximum activity indicator, activity category score indicator, category denominator indicator, category numerator indicator, activity counting time interval indicator, activity counting time interval indicator, client preference indicator, composite score indicator, composite score data object creation time indicator, adjustable indicator, and current time indicator, details of which are described herein.

In the present disclosure, the term “client profile data object” may refer to a type of data object that comprises, represents, indicates, and/or is associated with data, files, and/or information of a user in an example activity score data object and/or composite score data object generating platform/system. Such data, files, and/or information include, but not limited to, data, files, and/or information associated with the health of the user, data, files, and/or information associated with the activities of the user, and/or the like.

In the present disclosure, the term “client identifier indicator” may refer to a type of indicator or identifier that uniquely identifies data and/or information related to a user and stored in an example activity score data object and/or composite score data object generating platform/system, such as, but not limited to, a client profile data object associated with the user. In some embodiments, the client identifier indicator may comprise one or more alphabetical characters, one or more numerical characters, one or more alphanumeric characters, one or more ASCII texts, one or more pointers, one or more memory addresses, and/or the like.

In the present disclosure, the term “health score data object” may refer to a type of data object that comprises, represents, indicates, and/or is associated with a score describing an overall health level associated with a user (e.g. associated with a client identifier indicator), where the user may be associated with an example activity score data object and/or composite score data object generating platform/system. For example, an example health score data object may comprise data and/or information that indicates, represents, describes a health state of physical, mental and/or social well-being associated with the user (e.g. associated with a client identifier indicator).

In some embodiments, an example health score data object may comprise or be associated with a health score indicator. In the present disclosure, the term “health score indicator” may refer to a type of indicator that comprises, represents, describes, indicates, and/or is associated with an overall health score associated with a user (e.g. associated with a client identifier indicator) that is received by, retrieved by, generated by, calculated by, determined by, and/or stored by an example activity score data object and/or composite score data object generating platform/system. For example, the health score indicator may be derived from users' health history and survey responses to present their relative health status compared to their peers. In some embodiments, the health score indicator may comprise one or more alphabetical characters, one or more numerical characters, one or more alphanumeric characters, one or more ASCII texts, one or more pointers, one or more memory addresses, and/or the like.

In some embodiments, an example health score indicator may provide a metrics that indicates an overall health level associated with the user (e.g. associated with a client identifier indicator), details of which are described herein. For example, the example health score indicator may provide a health score that ranges from 0 to 1. As described in detail herein, an example health score indicator may be generated for each of a plurality of categories (such as, but not limited to, diet, sleep, etc.) that indicates the user's health level in that category. In some embodiments, each of the plurality of categories may be associated with one or more subcategories, and weights are assigned to each subcategory. In some embodiments, these weights are specific to a user demographic group. In some embodiments, an example health score indicator may be calculated by averaging sub-category and category level scores.

In the present disclosure, the term “category indicator” may refer to a type of indicator that comprises, represents, describes, indicates, and/or is associated with a category of health and/or activity that is recorded, tracked and/or monitored by an example activity score data object and/or composite score data object generating platform/system. For example, a category indicator may indicate or represent a theme, a class, or/or a division associated with a user's health and/or activity. In some embodiments, the category indicator may comprise one or more alphabetical characters, one or more numerical characters, one or more alphanumeric characters, one or more ASCII texts, one or more pointers, one or more memory addresses, and/or the like. As an example, TABLE 1 below provides some example category indicators in an example activity score data object and/or composite score data object generating platform/system:

TABLE 1
EXAMPLE CATEGORY INDICATORS
Category Indicator

Clinical
Diet
Exercise
Sleep
State Of Mind

While the description above provides some examples of category indicators, it is noted that the scope of the present disclosure is not limited to the description above. Example embodiments of the present disclosure may provide additional and/or alternative category indicators, such as, but not limited to, biometrics.

In some embodiments, an example health score data object may comprise or be associated with a health category score indicator. In the present disclosure, the term “health category score indicator” may refer to a type of indicator that comprises, represents, describes, indicates, and/or is associated with a health score in a category (e.g. corresponding to a category indicator) and associated with a user (e.g. associated with a client identifier indicator) that is received by, retrieved by, generated by, calculated by, determined by, and/or stored by an example activity score data object and/or composite score data object generating platform/system. In some embodiments, an example health category score indicator may provide a metrics (e.g. a health category score) that indicates a health level that is associated with a user (e.g. associated with a client identifier indicator) in a particular category (according to a category indicator). In some embodiments, the health category score indicator may comprise one or more alphabetical characters, one or more numerical characters, one or more alphanumeric characters, one or more ASCII texts, one or more pointers, one or more memory addresses, and/or the like.

Continuing from the example above, TABLE 2 below provides some example category indicators and the corresponding health category score indicators associated with a user (e.g. associated with a client identifier indicator) of an example activity score data object and/or composite score data object generating platform/system:

TABLE 2
EXAMPLE CATEGORY INDICATORS AND EXAMPLE
HEALTH CATEGORY SCORE INDICATORS

| Category Indicator | Health Category Score Indicator |
|---|---|
| Clinical | 0.35 |
| Diet | 0.56 |
| Exercise | 0.13 |
| Sleep | 0.6 |
| State Of Mind | 0.85 |

While the description above provides some examples of health category score indicators, it is noted that the scope of the present disclosure is not limited to the description above.

In the present disclosure, the term “health category maximum indicator” may refer to a type of indicator that comprises, represents, describes, indicates, and/or is associated with a maximum health score of a category (e.g. corresponding to a category indicator). In some embodiments, the health category maximum indicator may comprise one or more alphabetical characters, one or more numerical characters, one or more alphanumeric characters, one or more ASCII texts, one or more pointers, one or more memory addresses, and/or the like.

In some embodiments, an example activity score data object and/or composite score data object generating platform/system may set the health category maximum indicator as numerical value one (1) for each category. In some embodiments, an example activity score data object and/or composite score data object generating platform/system may set the health category maximum indicator less than or more than one (1). In some embodiments, an example activity score data object and/or composite score data object generating platform/system may set the same health category maximum indicator for different categories. In some embodiments, an example activity score data object and/or composite score data object generating platform/system may set different health category maximum indicators for different categories.

In the present disclosure, the term “gap indicator” may refer to a type of indicator that comprises, represents, describes, indicates, and/or is associated with a difference between a health category score indicator and a health category maximum indicator in a category (e.g. corresponding to a category indicator). In some embodiments, the gap indicator may comprise one or more alphabetical characters, one or more numerical characters, one or more alphanumeric characters, one or more ASCII texts, one or more pointers, one or more memory addresses, and/or the like.

Continuing from the example above, TABLE 3 below provides some example category indicators, the corresponding health category score indicators, the corresponding health category maximum indicator, and the corresponding gap indicators associated with a user (e.g. associated with a client identifier indicator) of an example activity score data object and/or composite score data object generating platform/system:

TABLE 3
EXAMPLE CATEGORY INDICATORS, EXAMPLE
HEALTH CATEGORY SCORE INDICATORS,
EXAMPLE HEALTH CATEGORY MAXIMUM INDICATORS,
AND EXAMPLE GAP INDICATORS

| Category | Health Category | Health Category | Gap |
|---|---|---|---|
| Indicator | Score Indicator | Maximum Indicator | Indicator |
| Clinical | 0.35 | 1 | 0.65 |
| Diet | 0.56 | 1 | 0.44 |
| Exercise | 0.13 | 1 | 0.87 |
| Sleep | 0.6 | 0.9 | 0.2 |
| State Of Mind | 0.85 | 1 | 0.15 |

In the present disclosure, the term “decay indicator” or “discount indicator” may refer to a type of indicator that comprises, represents, describes, indicates, and/or is associated with a decrease rate or percentage of a health level associated with a user over a period of time (for example, due to the user not participating in any healthy activities). In some embodiments, the decay indicator or the discount indicator may comprise one or more alphabetical characters, one or more numerical characters, one or more alphanumeric characters, one or more ASCII texts, one or more pointers, one or more memory addresses, and/or the like.

In some embodiments, an example decay indicator may be calculated based at least in part on an exponential algorithm. For example, the following equation provides an example techniques for calculating an example decay indicator:

α_{c}=exp(−Gap_{c}×β+γ) Equation 1

In Equation 1, Gap_{c} is the gap indicator associated with a category indicator c, α_{c} is the decay indicator associated with the category indicator c. β and γ are adjustable indicators (as defined further herein). In some embodiments, β is set to 10, and γ is set to 3. In some embodiments, the value of β and/or the value of γ may be set to other values.

While the description above provides an example calculation of a decay indicator, it is noted that the scope of the present disclosure is not limited to the description above. In some examples, an example decay indicator may be calculated based at least in part on one or more additional and/or alternative algorithms.

In the present disclosure, the term “adjustable indicator” may refer to a type of indicator that comprises, represents, describes, indicates, and/or is associated with a data value, a data field, a data unit or a data element that can be set by and/or is adjustable by an example activity score data object and/or composite score data object generating platform/system or by a user. In some embodiments, the adjustable indicator may comprise one or more alphabetical characters, one or more numerical characters, one or more alphanumeric characters, one or more ASCII texts, one or more pointers, one or more memory addresses, and/or the like.

In the present disclosure, the term “client activity data object” may refer to a type of data object that comprises, represents, indicates, and/or describes various activities that a user (e.g. associated with a client identifier indicator) has participated in and/or completed.

In some embodiments, an example client activity data object may comprise, represent, indicate, and/or describe, and/or be associated with one or more category indicators, which corresponding one or more categories of activities that a user has participated in or has completed, details of which are described herein.

Additionally, or alternatively, an example client activity data object may comprise, represent, indicate, and/or describe, and/or be associated with one or more type indicators, which corresponding one or more types of activities that a user has participated in or has completed, details of which are described herein.

Additionally, or alternatively, an example client activity data object may comprise, represent, indicate, and/or describe, and/or be associated with one or more actual activity indicators, which corresponding a number of actual activities that a user has participated in or has completed, details of which are described herein.

In some embodiments, based at least in part on the client activity data object, various embodiments of the present disclosure may generate an example activity score data object, details of which are described herein.

In the present disclosure, the term “activity score data object” may refer to a type of data object that comprises, represents, indicates, and/or is associated with an activity level associated with a user (e.g. associated with a client identifier indicator) of an example activity score data object and/or composite score data object generating platform/system. For example, an example activity score data object may comprise data and/or information that indicates, represents, describes levels of physical activity, mental activity and/or social activity that a user (e.g. associated with a client identifier indicator) has participated in and/or completed.

In some embodiments, an example activity score data object may comprise or be associated with an activity score indicator. In the present disclosure, the term “activity score indicator” may refer to a type of indicator that comprises, represents, describes, indicates, and/or is associated with an overall activity score associated with a user (e.g. associated with a client identifier indicator).

In the present disclosure, the term “type indicator” may refer to an indicator that comprises, represents, describes, indicates, and/or is associated with a type of activity that is recorded, tracked and/or monitored by an example activity score data object and/or composite score data object generating platform/system. In some embodiments, the type indicator may comprise one or more alphabetical characters, one or more numerical characters, one or more alphanumeric characters, one or more ASCII texts, one or more pointers, one or more memory addresses, and/or the like.

As described above, various embodiments of the present disclosure also provide a category indicator. As such, various activities can be categorized based at least in part on type indicators and categories indicators. In some embodiments, a category indicator may be associated with one or more type indicators. In some embodiments, a type indicator may be associated with one or more category indicators.

In some embodiments, a category indicator may indicate or represent a theme, a class, or/or a division associated with a user's activity, and a type indicator may indicate or represent a form, a format, or/or a style associated with a user's activity. Examples of type indicators may include, but not limited to, coaching, mission, articles, preventatives, user action, and/or the like. Examples of categories may include, but not limited to, biometrics, state of mind, diet, sleep, exercise, clinical compliance, and/or the like.

For example, an example activity score data object and/or composite score data object generating platform/system may be associated with a sleep coaching program “Sleepio” that helps users to improve their sleep. In this example, when a user participates in and/or completes the Sleepio program, this activity is associated with a type indicator indicating that it is a “coaching” type activity and a category indicator indicating that it is a “sleep” category.

In some embodiments, an activity may be associated with one or more category indicators. In some embodiments, an activity may be associated with one or more type indicators.

While the description above provides examples of category indicators and type indicators, it is noted that the scope of the present disclosure is not limited to the description above. In some examples, an example activity score data object and/or composite score data object generating platform/system may provide one or more additional and/or alternative category indicators, and/or one or more additional and/or alternative type indicators. Additionally, or alternatively, an activity described herein may be assigned to a different category indicator and/or a different type indicator as compared to the examples provided herein.

In the present disclosure, the term “point indicator” may refer to a type of indicator that comprises, represents, describes, indicates, and/or is associated with a reward point or an incentive that is set for and/or assigned to an activity once users have completed the activity. In some embodiments, the point indicator may comprise one or more alphabetical characters, one or more numerical characters, one or more alphanumeric characters, one or more ASCII texts, one or more pointers, one or more memory addresses, and/or the like.

In some embodiments, a point indicator is determined by an example activity score data object and/or composite score data object generating platform/system through machine learning algorithms. For example, a point indicator may be determined based at least in part on cost saving values by accomplishing an activity. In some embodiments, an example activity score data object and/or composite score data object generating platform/system may generate or assign point indicators to each category indicator and type indicator combination, and store the point indicators in a tabular format (for example, a data table) in a database.

In some embodiments, for a specific activity associated with a type indicator and a category indicator, there is a fixed value for a point indicator assigned to all the activities associated with the type indicator and the category indicator. TABLE 4 provides some of the example type indicators associated with the biometrics category indicator (which is not a complete list of activities) and their corresponding example point indicators:

TABLE 4
An EXAMPLE CATEGORY INDICATOR, EXAMPLE TYPE
INDICATORS, AND EXAMPLE POINT INDICATORS

| Category Indicator | Type Indicator | Point Indicator |
|---|---|---|
| Biometrics | Coaching | 100 |
| | Mission | 20 |
| | Article | 10 |

In some embodiments, an example activity score data object and/or composite score data object generating platform/system may assign point indicators to some activities in different ways. For example, various activities may be associated with clinical compliance (e.g. a clinical compliance category indicator) while also associated with different category indicators. In this example, the “clinical compliance” indicator is different from other category indicators, in that each activity is assigned different point indicators regardless of their category indicators. TABLE 5 provides an example of point indicators associated with some of the example activities associated with clinical compliance (which is not a complete list of activities):

TABLE 5
EXAMPLE CLINICAL COMPLIANCE CATEGORY, EXAMPLE
ACTIVITIES, AND EXAMPLE POINT INDICATORS

| Category Indicator | Activity | Point Indicator |
|---|---|---|
| Clinical Compliance | Flu Shot | 100 |
| | Annual Exam | 70 |
| | Diabetes Screening | 45 |

While the description above provides examples of point indicators, it is noted that the scope of the present disclosure is not limited to the description above. In some examples, an example activity score data object and/or composite score data object generating platform/system may assign different point indicators to one or more activities noted above.

In some embodiments, an example activity score data object may comprise or be associated with an activity type score indicator. In some embodiments, an example activity category score data object may comprise or be associated with an activity type score indicator. In the present disclosure, the term “activity type score indicator” may refer to a type of indicator that comprises, represents, describes, indicates, and/or is associated with an activity score that is calculated based at least in part on one or more activities in a particular category (e.g. corresponding to a category indicator) and a particular type (e.g. corresponding to a type indicator) that a user (e.g. associated with a client identifier indicator) has participated in and/or completed. In some embodiments, an example health category score indicator may provide a metrics that indicates a level of activity or activities that is a user (e.g. associated with a client identifier indicator) has participated in and/or completed, and such activity or activities are in a particular category (according to a category indicator) and associated with a particular type (according to a type indicator). In some embodiments, the activity type score indicator may comprise one or more alphabetical characters, one or more numerical characters, one or more alphanumeric characters, one or more ASCII texts, one or more pointers, one or more memory addresses, and/or the like

In the present disclosure, the term “type denominator indicator” may refer to a type of indicator that comprises, represents, describes, indicates, and/or is associated with a goal of activity or activities in a particular category (e.g. corresponding to a category indicator) and a particular type (e.g. corresponding to a type indicator) that a user (e.g. associated with a client identifier indicator) should participate in and/or complete. In some embodiments, a type denominator indicator may be calculated based at least in part on a health level associated with the user, details of which are described herein. In some embodiments, the type denominator indicator may comprise one or more alphabetical characters, one or more numerical characters, one or more alphanumeric characters, one or more ASCII texts, one or more pointers, one or more memory addresses, and/or the like.

In the present disclosure, the term “type numerator indicator” may refer to a type of indicator that comprises, represents, describes, indicates, and/or is associated with an accomplishment of activity or activities in a particular category (e.g. corresponding to a category indicator) and a particular type (e.g. corresponding to a type indicator) that a user (e.g. associated with a client identifier indicator) has participated in and/or completed. In some embodiments, the type numerator indicator may comprise one or more alphabetical characters, one or more numerical characters, one or more alphanumeric characters, one or more ASCII texts, one or more pointers, one or more memory addresses, and/or the like.

In the present disclosure, the term “activity counting time interval indicator” may refer to a type of indicator that comprises, represents, describes, indicates, and/or is associated with a time window for counting the number of activities that a user has completed. In some embodiments, the activity counting time interval indicator may be determined based at least in part on a category indicator and/or a type indicator associated with an activity. In some embodiments, the activity counting time interval indicator may comprise one or more alphabetical characters, one or more numerical characters, one or more alphanumeric characters, one or more ASCII texts, one or more pointers, one or more memory addresses, and/or the like.

For example, an example activity score data object and/or composite score data object generating platform/system may be set to count the number of activities that a user has completed over the past 3 months, and the activity counting time interval indicator is set to 3 months. In some embodiments, an example activity score data object and/or composite score data object generating platform/system may set the activity counting time interval indicator to other values.

In the present disclosure, the term “actual activity indicator” may refer to a type of indicator that comprises, represents, describes, indicates, and/or is associated with a number of times that a user (e.g. associated with a client identifier indicator) has completed a certain activity. In some examples, the number of activities counted by the actual activity indicator are in a particular category (e.g. corresponding to a category indicator) and a particular type (e.g. corresponding to a type indicator). In some examples, the number of activities is determined based at least in part on an activity counting time interval indicator, details of which are described herein. In some embodiments, the actual activity indicator may comprise one or more alphabetical characters, one or more numerical characters, one or more alphanumeric characters, one or more ASCII texts, one or more pointers, one or more memory addresses, and/or the like.

For example, if a user has attended three activities associated with the exercise category indicator and the coaching type indicator (for example, attended three exercise coaching sessions), the actual activity indicator associated with the exercise category indicator and the coaching type indicator may indicate a numerical value of three (3).

In the present disclosure, the term “maximum activity indicator” may refer to a type of indicator that comprises, represents, describes, indicates, and/or is associated with a maximum or available number of activities in a particular category (e.g. corresponding to a category indicator) and a particular type (e.g. corresponding to a type indicator) that a user can participate in or complete. In some embodiments, an example activity score data object and/or composite score data object generating platform/system may set maximum activity indicators. In some embodiments, the maximum activity indicator may comprise one or more alphabetical characters, one or more numerical characters, one or more alphanumeric characters, one or more ASCII texts, one or more pointers, one or more memory addresses, and/or the like.

Continuing from the example above, an example maximum activity indicator associated with the exercise category indicator and the coaching type indicator may be set to five (5), which indicates the maximum number of exercise coaching sessions that a user can attend is five.

In some embodiments, an example activity score data object may comprise or be associated with an activity category score indicator. In the present disclosure, the term “activity category score indicator” may refer to a type of indicator that comprises, represents, describes, indicates, and/or is associated with an activity score that is calculated based at least in part on one or more activities in a particular category (e.g. corresponding to a category indicator) has participated in and/or completed. In some embodiments, an example activity category score indicator may provide a metrics that indicates a level of activity or activities that is a user (e.g. associated with a client identifier indicator) has participated in and/or completed, and such activity or activities are in a particular category (according to a category indicator). In some embodiments, an example activity category score indicator of a category indicator may be generated based at least in part on aggregating type numerator indicators and type denominator indicators associated with type indicators under the category indicator, details of which are described herein. In some embodiments, the activity category score indicator may comprise one or more alphabetical characters, one or more numerical characters, one or more alphanumeric characters, one or more ASCII texts, one or more pointers, one or more memory addresses, and/or the like.

In the present disclosure, the term “category denominator indicator” may refer to a type of indicator that comprises, represents, describes, indicates, and/or is associated with a goal of activity or activities in a particular category (e.g. corresponding to a category indicator) that a user (e.g. associated with a client identifier indicator) should participate in and/or completed. In some embodiments, a category denominator indicator may be calculated based at least in part on a health level associated with the user, details of which are described herein. In some embodiments, the category denominator indicator may comprise one or more alphabetical characters, one or more numerical characters, one or more alphanumeric characters, one or more ASCII texts, one or more pointers, one or more memory addresses, and/or the like.

In the present disclosure, the term “category numerator indicator” may refer to a type of indicator that comprises, represents, describes, indicates, and/or is associated with an accomplishment of activity or activities in a particular category (e.g. corresponding to a category indicator) that a user (e.g. associated with a client identifier indicator) has participated in and/or completed. In some embodiments, the category numerator indicator may comprise one or more alphabetical characters, one or more numerical characters, one or more alphanumeric characters, one or more ASCII texts, one or more pointers, one or more memory addresses, and/or the like.

In the present disclosure, the term “client preference indicator” may refer to a type of indicator that comprises, represents, describes, indicates, and/or is associated with one or more preferences associated with a user (e.g. associated with a client identifier indicator), including, but not limited to, activities that the user likes to participate, activities that the user does not like to participate, a priority of activities for the user, and/or the like. In some embodiments, the client preference indicator may comprise one or more alphabetical characters, one or more numerical characters, one or more alphanumeric characters, one or more ASCII texts, one or more pointers, one or more memory addresses, and/or the like.

In the present disclosure, the term “activity recommendation data object” may refer to a type of data object that comprises, represents, indicates, and/or is associated with one or more activities recommended by an example activity score data object and/or composite score data object generating platform/system for a user (e.g. associated with a client identifier indicator) to participate. In some embodiments, an example activity score data object and/or composite score data object generating platform/system may generate the activity recommendation data object based at least in part on the health score data object and the activity score data object, details of which are described herein.

In the present disclosure, the terms “composite score data object” or “health activity score data object” refer to a type of data object that comprises, represents, indicates, and/or is associated with an overall health and activity level associated with a user (e.g. associated with a client identifier indicator) in an example activity score data object and/or composite score data object generating platform/system. For example, an example composite score data object may comprise data and/or information that indicates, represents, describes an overall health state and activity level associated with a user (e.g. associated with a client identifier indicator). In some embodiments, an example composite score data object may be generated based at least in part on a health score data object and an activity score data object associated with a user (e.g. associated with a client identifier indicator), details of which are described herein.

In some embodiments, an example health score data object may comprise or be associated with a composite score indicator or a health activity score indicator. In the present disclosure, the terms “composite score indicator” or “health activity score indicator” refer to a type of indicator that comprises, represents, describes, indicates, and/or is associated with an overall composite score or an overall health activity score associated with a user (e.g. associated with a client identifier indicator). In some embodiments, the composite score indicator or the health activity score indicator may comprise one or more alphabetical characters, one or more numerical characters, one or more alphanumeric characters, one or more ASCII texts, one or more pointers, one or more memory addresses, and/or the like.

In the present disclosure, the term “composite score data object creation time indicator” may refer to a type of indicator that comprises, represents, describes, indicates, and/or is associated with a time point, a time stamp, and/or a time code of when a composite score data object is created or generated. In some embodiments, the composite score data object creation time indicator may comprise one or more alphabetical characters, one or more numerical characters, one or more alphanumeric characters, one or more ASCII texts, one or more pointers, one or more memory addresses, and/or the like.

In the present disclosure, the term “activity score data object creation time indicator” may refer to a type of indicator that comprises, represents, describes, indicates, and/or is associated with a time point, a time stamp, and/or a time code of when an activity score data object is created or generated. In some embodiments, the activity score data object creation time indicator may comprise one or more alphabetical characters, one or more numerical characters, one or more alphanumeric characters, one or more ASCII texts, one or more pointers, one or more memory addresses, and/or the like.

In the present disclosure, the term “current time indicator” may refer to a type of indicator that comprises, represents, describes, indicates, and/or is associated with a current time in the format of a time point, a time stamp, and/or a time code. For example, the current time indicator may be determined based at least in part on an example activity score data object and/or composite score data object generating platform/system's notion of the current time and date so that various services of the example activity score data object and/or composite score data object generating platform/system can calibrate their respective clocks and otherwise access a common time. The current time is typically measured by a system clock or by a network associated device that is designated as the system clock. For example, an example activity score data object and/or composite score data object generating platform/system may utilize a time server that reads the actual time from a reference clock (e.g. a connected radio clock, an atomic clock, another time server on the network or the Internet) and distributes the time data to other components of the an example activity score data object and/or composite score data object generating platform/system. In some embodiments, the current time indicator may comprise one or more alphabetical characters, one or more numerical characters, one or more alphanumeric characters, one or more ASCII texts, one or more pointers, one or more memory addresses, and/or the like.

In the present disclosure, the term “activity update time interval indicator” may refer to a type of indicator that comprises, represents, describes, indicates, and/or is associated with a time interval for updating an activity score data object or a composite score data object associated with a user. In some embodiments, the activity counting time interval indicator may comprise one or more alphabetical characters, one or more numerical characters, one or more alphanumeric characters, one or more ASCII texts, one or more pointers, one or more memory addresses, and/or the like.

For example, an example activity score data object and/or composite score data object generating platform/system may be set to update activity score data objects every 24 hours, and the activity update time interval indicator is set to 24 hours. In some embodiments, an example activity score data object and/or composite score data object generating platform/system may set the activity update time interval indicator to other values.

In the present disclosure, the term “activity update data object” may refer to a type of data object that comprises, represents, indicates, and/or is associated with updates on additional activities that a user (e.g. associated with a client identifier indicator) has participated in and/or completed after an actual activity indicator, a type numerator indicators, a type denominator indicators, an activity category score indicator, an activity score data object, and/or a composite score data object has been determined or generated. In some embodiments, based at least in part on the activity update data object, an updated actual activity indicator, an updated type numerator indicators, an updated type denominator indicators, an updated activity category score indicator, an updated activity score data object, and/or an updated composite score data object may be generated, details of which are described herein.

In the present disclosure, the term “score-based action” may refer to a computer-based operation associated with a data object.

In the present disclosure, the term “user interface” may refer to a space where interactions between a user and a computing device may occur. For example, an example user interface may be a graphical user interface that is rendered on a display of a client computing entity.

### d. Exemplary Techniques for Generation of a Composite Score Data Object

As described above, there are technical challenges, deficiencies and problems associated with data analytics systems, and various example embodiments of the present overcome such challenges. Referring now to FIG. 4, an example method 400 of generating one or more composite score data objects and performing at least one score-based action based at least in part on the composite score data object that overcomes various technical challenges in accordance with embodiments of the present disclosure is illustrated. For example, the example method 400 generates an activity score data object based at least in part on a health score data object, therefore establishing a data connection between data/information associated with a user's health and data/information associated with a user's activity. The example method 400 further generates a composite score data object based at least in part on a health score data object and an activity score data object, and performs one or more score-based actions based at least in part on the composite score data object (for example, generating activity recommendation data objects to encourage users to participate in healthy activities).

As shown in FIG. 4, the example method 400 starts at step/operation 402. Subsequent to step/operation 402, the example method 400 proceeds to step/operation 404. At step/operation 404, a computing entity (such as the data object computing entity 105 described above in connection with FIG. 1 and FIG. 2) may include means (such as the processing element 205 of the data object computing entity 105 described above in connection with FIG. 2) to retrieve a client profile data object associated with a health score data object and at least one client activity data object.

In some embodiments, the processing element retrieves a client profile data object based at least in part on a client identifier indicator. For example, the processing element may retrieve the client profile data object from a database or a data storage device in an example activity score data object and/or composite score data object generating platform/system by transmitting a database query to the database or the data storage device. In some embodiments, the database query comprises a client identifier indicator, which uniquely identifies data and/or information related to a user of the example activity score data object and/or composite score data object generating platform/system as described above.

In some embodiments, the client profile data object is associated with a health score data object and at least one client activity data object. For example, the client profile data object, the health score data object, and the at least one client activity data object may be associated with the same client identifier indicator.

As described above, the health score data object may refer to a type of data object that comprises, represents, indicates, and/or is associated with a health level associated with a user in an example activity score data object and/or composite score data object generating platform/system. For example, the health score data object may comprise a health score indicator that indicates a health score associated with the user (e.g. associated with the client identifier indicator).

In some embodiments, the at least one client activity data object may comprise or be associated with one or more category indicators and/or one or more type indicators, which may correspond to one or more categories and/or one more types of activities that a user (e.g. associated with the client identifier indicator) has participated in. Additionally, or alternatively, the at least one client activity data object may comprise or be associated with one or more actual activity indicators, which may indicate the actual number of times that a user has completed a certain activity that is associated with a particular category indicator and a particular type indicator.

As an example, TABLE 6 below provides an example illustration of an example client activity data object that is in a tabular format:

TABLE 6
EXAMPLE CLIENT ACTIVITY DATA OBJECT

| Category Indicator | Type Indicator | Actual Activity Indicator |
|---|---|---|
| Exercise | Coaching | 2 |
| Sleep | Article | 3 |
| | Coaching | 1 |

In the TABLE 6 example above, the example client activity data object indicates that the user has completed exercise coaching activity twice, read articles on sleep three times, and completed sleep coaching activity once.

While the description above provides an example of a client activity data object, it is noted that the scope of the present disclosure is not limited to the description above. In some examples, an example client activity data object may comprise one or more additional and/or alternative category indicators, type indicators, and/or actual activity indicators.

Referring back to FIG. 4, subsequent to and/or in response to step/operation 404, the example method 400 proceeds to step/operation 406. At step/operation 406, a computing entity (such as the data object computing entity 105 described above in connection with FIG. 1 and FIG. 2) may include means (such as the processing element 205 of the data object computing entity 105 described above in connection with FIG. 2) to determine a plurality of point indicators based at least in part on the at least one client activity data object.

As described above, a point indicator may refer to a type of indicator that comprises, represents, describes, indicates, and/or is associated with a reward point or an incentive that is set for and/or assigned to an activity once users have completed the activity. In some embodiments, the plurality of point indicators is associated with a plurality of category indicators and a plurality of type indicators.

In some embodiments, the client activity data object associated with client profile data object retrieved at step/operation 404 may comprise one or more category indicators and/or one or more type indicators. In some embodiments, based at least in part on these one or more category indicators and/or one or more type indicators, the processing element may determine the plurality of point indicators.

For example, the processing element may retrieve a data table that indicates a corresponding point indicator associated with a particular category indicator and a particular type indicator. Continuing from the example, based at least in part on the example client activity data object indicating that the user has completed exercise coaching activity, the processing element may retrieve the data table and determine that a point indicator for an activity associated with an exercise category indicator and a coaching type indicator is 50. Based at least in part on the example client activity data object indicating that the user has read articles on sleep, the processing element may retrieve the data table and determine that a point indicator for an activity associated with a sleep category indicator and an article type indicator is 20. Based at least in part on the example client activity data object indicating that the user has completed sleep coaching activity, the processing element may retrieve the data table and determine that a point indicator for an activity associated with a sleep category indicator and a coaching type indicator is 80.

Referring back to FIG. 4, subsequent to and/or in response to step/operation 406, the example method 400 proceeds to step/operation 408. At step/operation 408, a computing entity (such as the data object computing entity 105 described above in connection with FIG. 1 and FIG. 2) may include means (such as the processing element 205 of the data object computing entity 105 described above in connection with FIG. 2) to generate an activity score data object based at least in part on a plurality of point indicators and a health score data object.

In some embodiments, through step/operation 408, various embodiments of the present invention introduce techniques for generating activity scores that are configured to be used to recommend activities to an end-user of a device. For example, various embodiments of the present invention introduce techniques for generating activity scores based on point indicators associated with an activity, where each point indicator may be associated with a combination of an activity category indicator and an activity type indicator. By utilizing the noted techniques, various embodiments of the present invention enable techniques for expedited and relatively non-resource-intensive generation of activity scores that enable generating such scores on end-user client devices, rather than on server systems. This in turn reduces the need for client-server communications in order to generate activity scores, which in turn improves network efficiency and network reliability of distributed user activity monitoring systems.

In some embodiments, the processing element generates an activity score data object associated with the client profile data object based at least in part on the plurality of category indicators as indicated by the client activity data object associated with the client profile data object retrieved at step/operation 404, the plurality of type indicators as indicated by the client activity data object associated with the client profile data object retrieved at step/operation 404, the plurality of point indicators as determined at step/operation 406, and the health score data object associated with the client profile data object retrieved at step/operation 404.

For example, the processing element may generate a type numerator indicator and a type denominator indicator for each of the type indicators as indicated by the client activity data object associated with the client profile data object retrieved at step/operation 404. As described above, the type denominator indicator describes a goal of activity or activities in a particular category and a particular type that a user should participate in and/or complete, and the type numerator indicator describes activity or activities in a particular category and a particular type that a user has participated in and/or completed.

Continuing from the example shown in TABLE 6 above, the processing element may calculate a type numerator indicator and a type denominator indicator associated with the sleep category indicator and the article type indicator. Additionally, the processing element may calculate a type numerator indicator and a type denominator indicator associated with the sleep category indicator and the coaching type indicator. Similarly, the processing element may calculate a type numerator indicator and a type denominator indicator for each type indicator under the sleep category indicator that is associated with activities where a user has participated in or completed.

Additionally, or alternatively, the processing element may generate an activity category score indicator associated with a category indicator based at least in part on the one or more type numerator indicators and type denominator indicators that are associated with one or more type indicators under the same category indicator.

Continuing from the example above, the processing element may calculate a first type numerator indicator and a first type denominator indicator associated with the sleep category indicator and the article type indicator, and a second type numerator indicator and a second type denominator indicator associated with the sleep category indicator and the coaching type indicator. The processing element may calculate the activity category score indicator associated with the sleep category indicator based at least in part on the first type numerator indicator, the first type denominator indicator, the second type numerator indicator and the second type denominator indicator.

Additionally, or alternatively, the processing element may generate an activity score indicator for the activity score data object based at least in part on the one or more activity category score indicators that are associated with one or more category indicators as indicated by the client activity data object associated with the client profile data object retrieved at step/operation 404.

Continuing from the example above, the processing element may calculate a first activity category score indicator associated with the sleep category indicator, and a second activity category score indicator associated with the diet category indicator. The processing element may calculate the activity score indicator of the activity score data object based at least in part on the first activity category score indicator and the second activity category score indicator.

Additional details associated with generating type numerator indicators, type denominator indicators, activity category score indicators, and activity score indicators for activity score data objects are described herein.

Referring back to FIG. 4, subsequent to and/or in response to step/operation 408, the example method 400 proceeds to step/operation 410. At step/operation 410, a computing entity (such as the data object computing entity 105 described above in connection with FIG. 1 and FIG. 2) may include means (such as the processing element 205 of the data object computing entity 105 described above in connection with FIG. 2) to generate a composite score data object based at least in part on the health score data object and the activity score data object.

In some embodiments, the processing element generates a composite score data object based at least in part on the health score data object associated with the client profile data object retrieved at step/operation 404 and the activity score data object generated at step/operation 410. In some embodiments, the composite score data object is associated with the client profile data object.

As described above, the composite score data object may be generated based at least in part on a health score data object and an activity score data object associated with a user. For example, the composite score data object may comprise a composite score indicator that is calculated based at least in part on a health score indicator associated with the health score data object and an activity score indicator associated with the activity score data object.

In some embodiments, the composite score indicator may be calculated based at least in part on a weighted sum of the health score indicator and the activity score indicator as shown in the following equation:

CompositeScore=ω_{A}×ActivityScore+ω_{H}×HealthScore Equation 2

In the above Equation 2, CompositeScore is the composite score indicator, ActivityScore is the activity score indicator of the activity score data object generated at step/operation 410, and HealthScore is the health score indicator of the health score data object associated with the client profile data object retrieved at step/operation 404. ω_{A} is the weight of the activity score indicator in the weighted sum calculation, and ω_{H} is the weight of the health score indicator in the weighted sum calculation.

In various embodiments of the present disclosure, the weights ω_{A} and ω_{H} can be calculated in various ways. As an example, the weights ω_{A} and ω_{H} can be calculated based at least in part on the following equations:

$${ω}_{A} = \frac{{Median}_{ActivityScore}}{{Median}_{ActivityScore} + {Median}_{HealthScore}} \qquad Equation 3$$

$${ω}_{H} = \frac{{Median}_{HealthScore}}{{Median}_{ActivityScore} + {Median}_{HealthScore}} \qquad Equation 4$$

In the above Equations 3-4, Median_{ActivityScore} is a median of health category score indicator(s) of the health score data object associated with the client profile data object retrieved at step/operation 404. Median_{ActivityScore} is a median of activity category score indicator(s) associated with the activity score data object generated at step/operation 408.

While the description above provides an example calculation of a composite score indicator, it is noted that the scope of the present disclosure is not limited to the description above. In some examples, an example composite score indicator may be calculated based at least in part on one or more additional and/or alternative algorithms.

Referring back to FIG. 4, subsequent to and/or in response to step/operation 410, the example method 400 proceeds to step/operation 412. At step/operation 412, a computing entity (such as the data object computing entity 105 described above in connection with FIG. 1 and FIG. 2) may include means (such as the processing element 205 of the data object computing entity 105 described above in connection with FIG. 2) to perform at least one score-based action based at least in part on the composite score data object and the client profile data object.

In some embodiments, the processing element may render the composite score data object on a user interface associated with a client computing entity. For example, the user interface may be a graphical user interface that is rendered on a display of the client computing entity.

In some embodiments, the processing element may further render a breakdown of the composite score indicator of the composite score data object according to category indicators. For example, users may be presented with a total composite score and a breakdown of the score by different categories on the user interface.

In some embodiments, the processing element may generate and render at least one activity recommendation data object on a user interface based at least in part on the composite data object. For example, the activity recommendation data object may include a list of actions users are able to take to improve their health. In some embodiments, the at least one activity recommendation data object is generated based at least in part on the users' preference and ability to engage as provided in the client preference indicator associated with the user.

In some embodiments, when a user takes an action, the processing element updates the corresponding composite score data object (e.g. increase the composite score indicator) to reflect the user's immediate engagement in healthy activities. In some embodiments, the processing element may update the composite score data object (e.g. decrease the composite score indicator) if a user disengages from healthy activities after a certain period of time. In some embodiments, the processing element may generate texts and/or images on the user interface that explain the change in the composite score data object.

Referring back to FIG. 4, subsequent to step/operation 412, the example method 400 proceeds to step/operation 414 and ends.

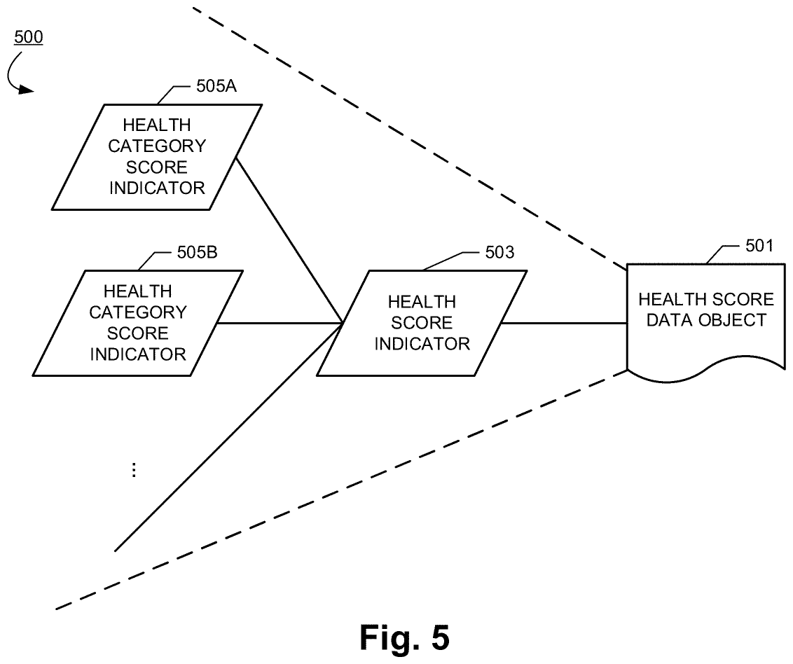

As described above, an example health score data object may comprise a health category score indicator, and the health category score indicator may be associated with one or more category indicators. Referring now to FIG. 5, an example diagram 500 illustrating example data relationships between a health score data object 501, a health score indicator 503, a health category score indicator 505A, and a health category score indicator 505B associated with a client identifier indicator is provided.

In the example shown in FIG. 5, each of the health category score indicator 505A and the health category score indicator 505B is associated with a corresponding category indicator. For example, TABLE 7 below provides example category indicators and example health category score indicators associated with a client identifier indicator that correspond to each of the example category indicators:

TABLE 6
EXAMPLE CATEGORY INDICATORS AND
HEALTH CATEGORY SCORE INDICATORS

| Reference | Category | Health Category |
|---|---|---|
| Number | Indicator | Score Indicator |
| 505A | Biometrics | 0.35 |
| 505B | Diet | 0.56 |

While the description above provides some examples of category indicators and health category score indicators, it is noted that the scope of the present disclosure is not limited to the description above.

In some embodiments, the health score indicator 503 may be calculated based at least in part on the health category score indicator associated with a client identifier indicator (such as, but not limited to, the health category score indicator 505A and the health category score indicator 505B). For example, the health score indicator 503 may be calculated based at least in part on a weighted sum of the health category score indicators.

As shown in FIG. 5, the health score data object 501 comprises the health score indicator 503. As such, the health score data object 501 comprises, represents, indicates, and/or is associated with an overall health level associated with a user (e.g. associated with a client identifier indicator) across different categories.

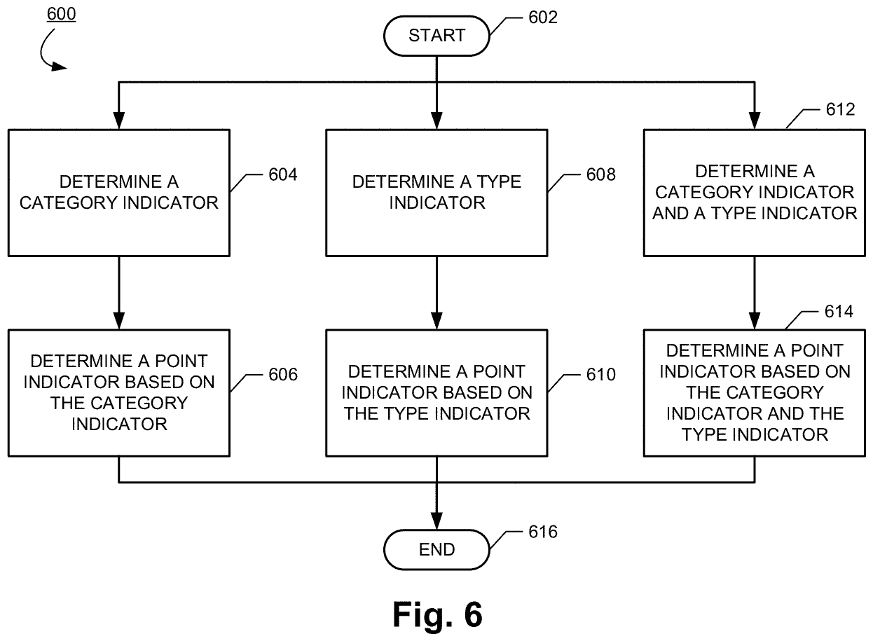

As described above, various examples of the present disclosure may determine a plurality of point indicators (for example, in connection with at least step/operation 406 described above in connection with FIG. 4). Referring now to FIG. 6, an example method 600 of determining a plurality of point indicators that overcomes various technical challenges in accordance with embodiments of the present disclosure is illustrated. In particular, the example method 600 illustrates various embodiments of determining a plurality of point indicators.

As shown in FIG. 6, the example method 600 starts at step/operation 602. Subsequent to step/operation 602, the example method 600 proceeds to step/operation 604. At step/operation 604, a computing entity (such as the data object computing entity 105 described above in connection with FIG. 1 and FIG. 2) may include means (such as the processing element 205 of the data object computing entity 105 described above in connection with FIG. 2) to determine a category indicator.

For example, as described above in connection with at least step/operation 404, the processing element may retrieve a client profile data object that is associated with at least one client activity data object. In some embodiments, the at least one client activity data object may indicate a plurality of category indicators that is associated with categories of activities that a user has participated in or completed. As an example, a category indicator of the plurality of category indicators may indicate that the category associated with the activity is an exercise category.

Referring back to FIG. 6, subsequent to and/or in response to step/operation 604, the example method 600 proceeds to step/operation 606. At step/operation 606, a computing entity (such as the data object computing entity 105 described above in connection with FIG. 1 and FIG. 2) may include means (such as the processing element 205 of the data object computing entity 105 described above in connection with FIG. 2) to determine a point indicator based at least in part on the category indicator determined at step/operation 604.

For example, the processing element determines the point indicator of the plurality of point indicators based at least in part on the category indicator of the plurality of category indicators indicated by at least one client activity data object. In some embodiments, the point indicator is associated with the category indicator determined at step/operation 604.

Continuing from the example above, the processing element may retrieve a data table that indicates a point indicator corresponding to each category indicator. Based at least in part on the data table, the processing element may determine a point indicator corresponding to the exercise category (for example, twenty (20)).

Referring back to FIG. 6, subsequent to step/operation 606, the example method 600 proceeds to step/operation 616 and ends.

As shown in FIG. 6, subsequent to step/operation 602, the example method 600 additionally, or alternatively, proceeds to step/operation 608. At step/operation 608, a computing entity (such as the data object computing entity 105 described above in connection with FIG. 1 and FIG. 2) may include means (such as the processing element 205 of the data object computing entity 105 described above in connection with FIG. 2) to determine a type indicator.

For example, as described above in connection with at least step/operation 404, the processing element may retrieve a client profile data object that is associated with at least one client activity data object. In some embodiments, the at least one client activity data object may indicate a plurality of type indicators that is associated with types of activities that a user has participated in or completed. As an example, a type indicator of the plurality of type indicators may indicate that the type associated with the activity is a coaching type.

Referring back to FIG. 6, subsequent to and/or in response to step/operation 608, the example method 600 proceeds to step/operation 610. At step/operation 610, a computing entity (such as the data object computing entity 105 described above in connection with FIG. 1 and FIG. 2) may include means (such as the processing element 205 of the data object computing entity 105 described above in connection with FIG. 2) to determine a point indicator based at least in part on the type indicator determined at step/operation 608.

For example, the processing element determines the point indicator of the plurality of point indicators based at least in part on the type indicator of the plurality of type indicators indicated by at least one client activity data object. In some embodiments, the point indicator is associated with the type indicator determined at step/operation 608.

Continuing from the example above, the processing element may retrieve a data table that indicates a point indicator corresponding to each type indicator. Based at least in part on the data table, the processing element may determine a point indicator corresponding to the coaching type (for example, twenty (20)).

Referring back to FIG. 6, subsequent to step/operation 610, the example method 600 proceeds to step/operation 616 and ends.

As shown in FIG. 6, subsequent to step/operation 602, the example method 600 additionally, or alternatively, proceeds to step/operation 612. At step/operation 612, a computing entity (such as the data object computing entity 105 described above in connection with FIG. 1 and FIG. 2) may include means (such as the processing element 205 of the data object computing entity 105 described above in connection with FIG. 2) to determine a category indicator and a type indicator.

For example, as described above in connection with at least step/operation 404, the processing element may retrieve a client profile data object that is associated with at least one client activity data object. In some embodiments, the at least one client activity data object may indicate a plurality of category indicators and a plurality of type indicators that are associated with categories and types of activities that a user has participated in or completed. As an example, a category indicator of the plurality of category indicators may indicate that the category associated with the activity is an exercise category, and a type indicator of the plurality of type indicators may indicate that the type associated with the activity is a coaching type.

Referring back to FIG. 6, subsequent to and/or in response to step/operation 612, the example method 600 proceeds to step/operation 614. At step/operation 614, a computing entity (such as the data object computing entity 105 described above in connection with FIG. 1 and FIG. 2) may include means (such as the processing element 205 of the data object computing entity 105 described above in connection with FIG. 2) to determine a point indicator based at least in part on the category indicator and the type indicator determined at step/operation 612.

For example, the processing element determines the point indicator of the plurality of point indicators based at least in part on the category indicator of the plurality of category indicators and the category indicator of the plurality of category indicators indicated by at least one client activity data object. In some embodiments, the point indicator is associated with the category indicator and the type indicator determined at step/operation 612.

Continuing from the example above, the processing element may retrieve a data table that indicates a point indicator corresponding to each category indicator and type indicator combination. Based at least in part on the data table, the processing element may determine a point indicator corresponding to the exercise category and the coaching type (for example, twenty (20)).

Referring back to FIG. 6, subsequent to step/operation 614, the example method 600 proceeds to step/operation 616 and ends.

### e. Exemplary Techniques for Generation of an Activity Score Data Object

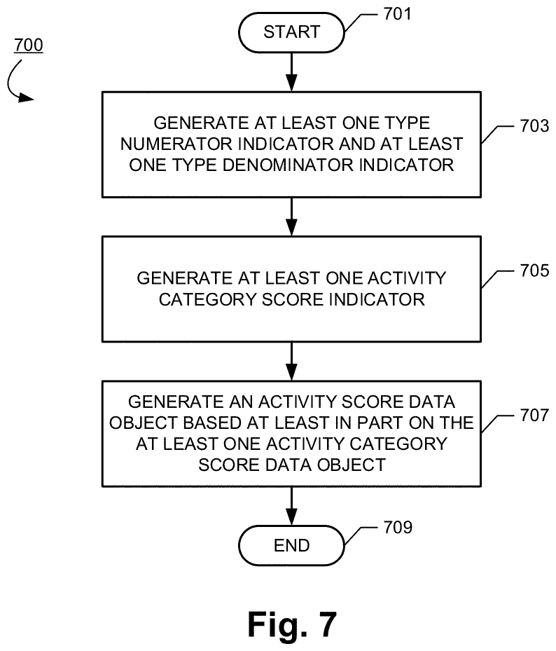

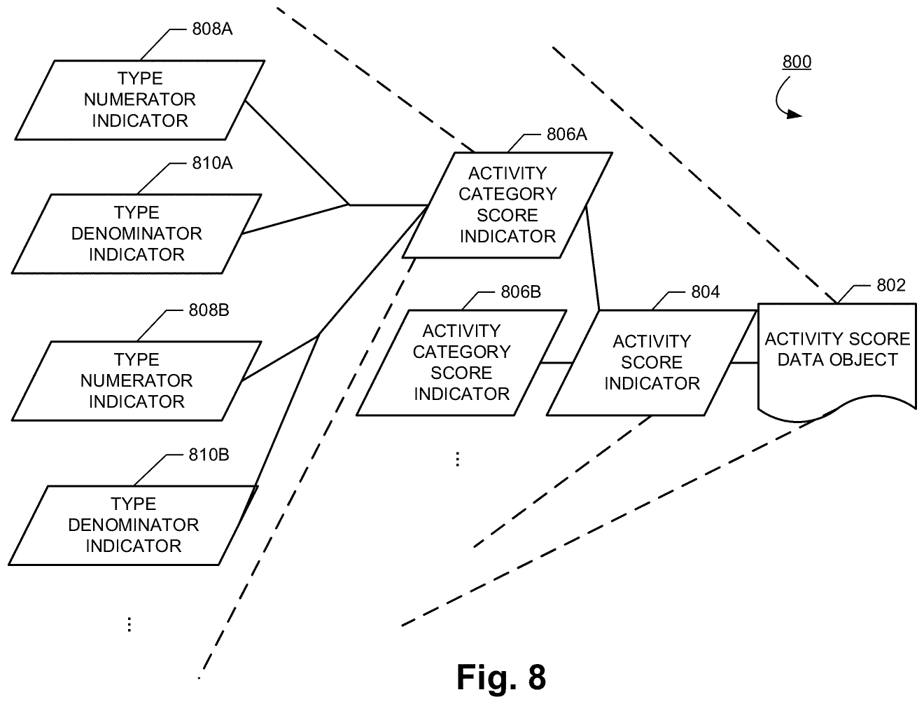

As described above, there are technical challenges, deficiencies and problems associated with data analytics systems, and various example embodiments of the present overcome such challenges. Referring now to FIG. 7 and FIG. 8, example diagrams in accordance with various embodiments of the present disclosure are illustrated. In particular, FIG. 7 illustrates an example method 700 of generating an activity score data object that overcomes various technical challenges in accordance with embodiments of the present disclosure. For example, the example method 700 generates an activity score data object based at least in part on one or more activity category score data objects associated with one or more category indicators that correspond to one or more categories of activities that the user has participated in or completed. FIG. 8 provides an example diagram 800 illustrating example data relationships between an activity score data object, an activity score indicator, activity category score indicators, type numerator indicators and type denominator indicators in accordance with embodiments of the present disclosure.

As shown in FIG. 7, the example method 700 starts at step/operation 701. Subsequent to step/operation 701, the example method 700 proceeds to step/operation 703. At step/operation 703, a computing entity (such as the data object computing entity 105 described above in connection with FIG. 1 and FIG. 2) may include means (such as the processing element 205 of the data object computing entity 105 described above in connection with FIG. 2) to generate at least one type numerator indicator and at least one type denominator indicator.

As described above, an example category indicator may be associated with one or more type indicators. For each category indicator, the processing element may determine one or more type indicators associated with types of activities that a user has participated in or completed (based at least in part on, such as but not limited to, at least one client activity data object associated with the user as described above), and may determine a health category score indicator associated with the category indicator.

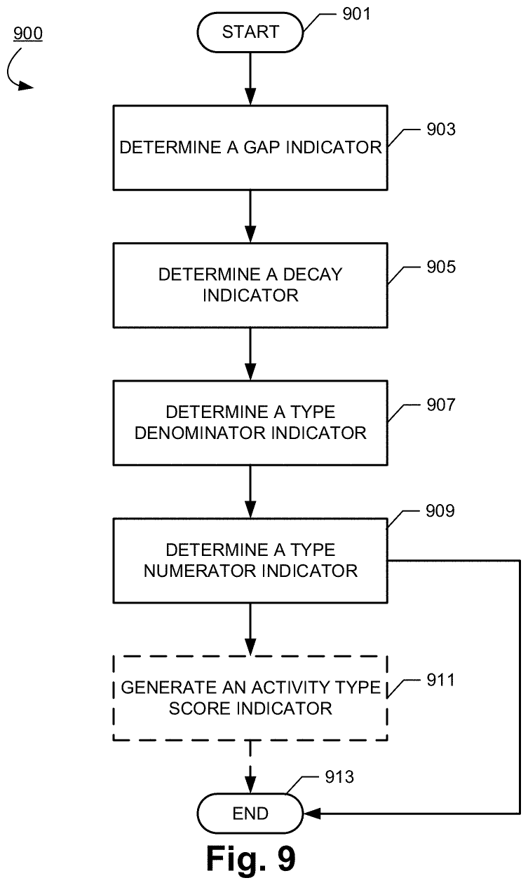

In some embodiments, for each category indicator, the processing element may generate the one or more type numerator indicators and one or more type denominator indicators for one or more type indicators under the category indicator based at least in part on the health score data object associated with the user (for example, a health category score indicator corresponding to the category indicator), one or more actual activity indicators associated with the one or more type indicators, and one or more point indicators associated with the one or more type indicators. Additional details of generating the at least one type numerator indicator and at least one type denominator indicator are described herein, including, but not limited to, those in connection with at least FIG. 9.

Referring now to FIG. 8, in some embodiments, the processing element may generate the type numerator indicator 808A and type denominator indicator 810A, both associated with a first type indicator that corresponds to a first type of activity that a user has participated in or completed. Additionally, the processing element may generate the type numerator indicator 808B and type denominator indicator 810B, both associated with a second type indicator that corresponds to a second type of activity that a user has participated in or completed. In such an example, both the first type indicator and the second type indicator are associated with the same category indicator.

For example, TABLE 8 below provides example type indicators associated with the same category indicator, as well as example type numerator indicators and type denominator indicators associated with example type indicators:

TABLE 8
EXAMPLE CATEGORY INDICATOR, TYPE
INDICATORS, TYPE NUMERATOR INDICATORS
AND TYPE NUMERATOR INDICATORS

| Reference | Category | Type | Indicator |
|---|---|---|---|
| Number | indicator | Indicator | Value |
| 808A | Sleep | Article | 277 |
| 810A | | | 595 |
| 808B | | Coaching | 310 |
| 810B | | | 460 |

In the TABLE 8 example above, the processing element generates an example type numerator indicator 808A and an example type denominator indicator 810A for the sleep activity category and the article activity type, and generates an example type numerator indicator 808B and an example type denominator indicator 810B for the sleep activity category and the coaching activity type.

Referring back to FIG. 7, subsequent to and/or in response to step/operation 703, the example method 700 proceeds to step/operation 705. At step/operation 705, a computing entity (such as the data object computing entity 105 described above in connection with FIG. 1 and FIG. 2) may include means (such as the processing element 205 of the data object computing entity 105 described above in connection with FIG. 2) to generate at least one activity category score indicator.

In some embodiments, the processing element may generate the at least one activity category score indicator based at least in part on the at least one type numerator indicator and at least one type denominator indicator generated at step/operation 703.

As described above, the processing element may generate one or more type numerator indicators and one or more type denominator indicators associated with one or more type indicators under the same category indicator. In some embodiments, the processing element may aggregate the one or more type numerator indicators and one or more type denominator indicators at the category level to generate at least one activity category score indicator.

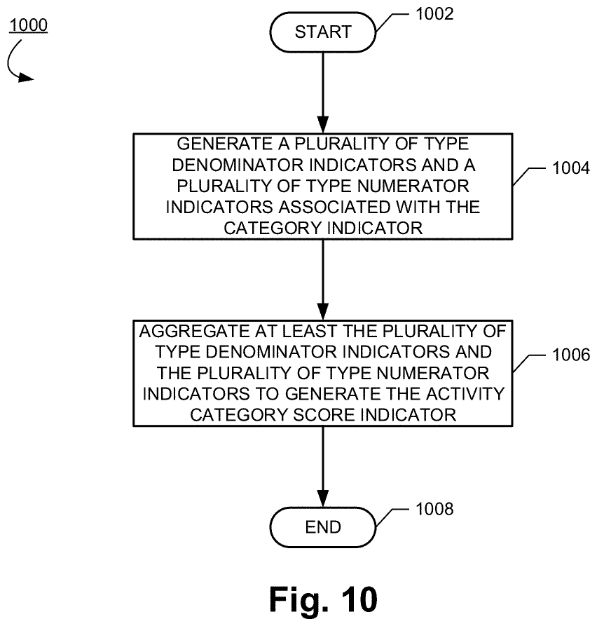

In some embodiments, as type numerator indicators and type denominator indicators are generated based at least in part on the health category score indicator associated with the category indicator, the processing element generates the activity category score indicator associated with the category indicator based at least in part on the health category score indicator. Additional details of generating activity category score indicators are described herein, including, but not limited to, those in connection with at least FIG. 10.

Referring now to FIG. 8, in some embodiments, the processing element may generate the activity category score indicator 806A based at least in part on aggregating the example type numerator indicator 808A, the example type denominator indicator 810A, the example type numerator indicator 808B, and the example type denominator indicator 810B. Continuing the TABLE 8 example above, the processing element generates an example activity category score indicator 806A for the sleep category based at least in part on aggregating the example type numerator indicator 808A and the example type denominator indicator 810A associated with the article type, as well as the example type numerator indicator 808B and the example type denominator indicator 810B associated with the coaching type. Similarly, the processing element may generate the activity category score indicator 806B for a different category indicator based at least in part on aggregating one or more type numerator indicators and one or more type denominator indicators that are associated with one or more type indicators under the same category indicator.

Referring back to FIG. 7, subsequent to and/or in response to step/operation 705, the example method 700 proceeds to step/operation 707. At step/operation 707, a computing entity (such as the data object computing entity 105 described above in connection with FIG. 1 and FIG. 2) may include means (such as the processing element 205 of the data object computing entity 105 described above in connection with FIG. 2) to generate an activity score data object based at least in part on the at least one activity category score data object.

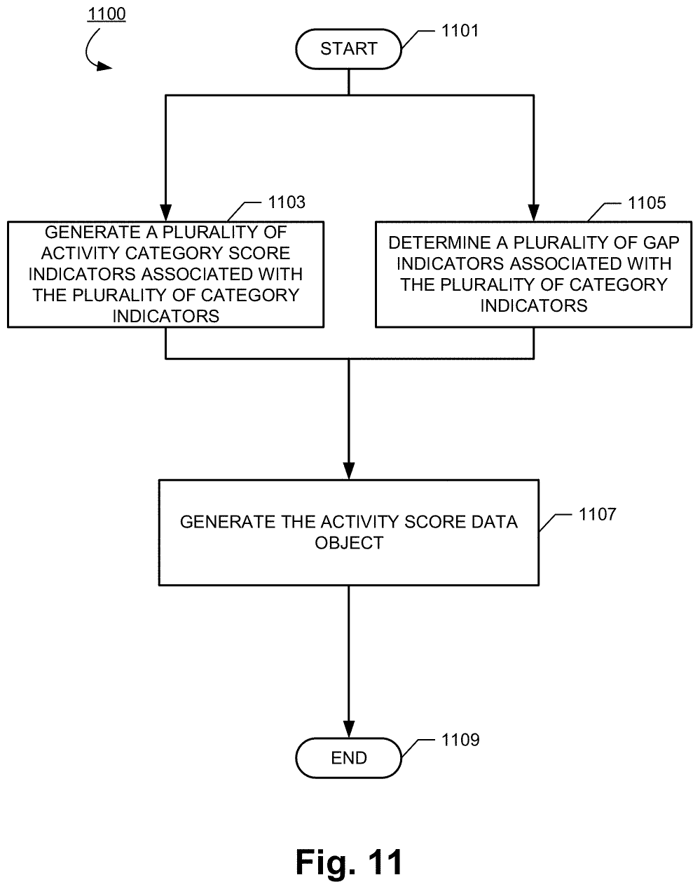

For example, the processing element may generate an activity score indicator for the activity score data object based at least in part on the at least one activity category score indicator generated at step/operation 705. As described above, the processing element may generate one or more activity category score indicators associated with one or more category indicators corresponding to categories of activities that a user has participated in or completed. In some embodiments, the processing element may aggregate the one or more activity category score indicators to generate an activity score indicator. Additional details of generating the activity score indicator are described herein, including, but not limited to, those in connection with at least FIG. 11.

Referring now to FIG. 8, in some embodiments, the processing element may generate the activity score indicator 804 for the activity score data object 802 based at least in part on aggregating at least the activity category score indicator 806A and the activity category score indicator 806B. For example, the processing element generates an example activity score indicator 804 based at least in part on aggregating the example activity category score indicators 806A associated with the sleep category indicator and the example activity category score indicators 806B associated with the exercise category.

Referring back to FIG. 7, subsequent to step/operation 707, the example method 700 proceeds to step/operation 709 and ends.

As described above, there are technical challenges, deficiencies and problems associated with data analytics systems, and various example embodiments of the present overcome such challenges. Referring now to FIG. 9, an example method 900 of generating an example type denominator indicator and an example type numerator indicator that overcomes various technical challenges in accordance with embodiments of the present disclosure is illustrated. For example, the example method 900 generate a type denominator indicator and a type numerator indicator associated with a type indicator and a category indicator based at least in part on a health category score indicator and a health category maximum indicator associated with category indicator, as well as a point indicator, a maximum activity indicator and an actual activity indicator associated with the type indicator. In some embodiments, the processing element may generate an activity type score indicator associated with a type indicator and a category indicator.

As shown in FIG. 9, the example method 900 starts at step/operation 901. Subsequent to step/operation 901, the example method 900 proceeds to step/operation 903. At step/operation 903, a computing entity (such as the data object computing entity 105 described above in connection with FIG. 1 and FIG. 2) may include means (such as the processing element 205 of the data object computing entity 105 described above in connection with FIG. 2) to determine a gap indicator.

In some embodiments, the processing element may generate one or more type numerator indicators and the one or more type denominator indicators associated with one or more type indicators under a category indicator. As such, for each category indicator, the processing element determines a gap indicator based at least in part on the health category score indicator associated with the category indicator and a health category maximum indicator associated with the category indicator. For example, the health category score indicator provides a health category score that indicates a health level in a particular category associated with a user, and the health category maximum indicator describes a maximum health score of that particular category. The gap indicator indicates a health “gap” of a user between the maximum health score and the user's current health score.

In some embodiments, for each category indicator, the processing element may calculate the gap indicator based at least in part on the following equation:

Gap_{c}=HealthMax_{c}−HealthScore_{c} Equation 5

In the above Equation 5, HealthMax_{c} is the health category maximum indicator associated with a category indicator c, HealthScore_{c} is the health category score indicator of a user associated with the category indicator c. In some embodiments, the health category maximum indicator is set to one (1), and the above equation may be represented as follows:

Gap_{c}=1−HealthScore_{c}

While the description above provides an example calculation of a gap indicator, it is noted that the scope of the present disclosure is not limited to the description above. In some examples, an example gap indicator may be calculated based at least in part on one or more additional and/or alternative algorithms.

Referring back to FIG. 9, subsequent to and/or in response to step/operation 903, the example method 900 proceeds to step/operation 905. At step/operation 905, a computing entity (such as the data object computing entity 105 described above in connection with FIG. 1 and FIG. 2) may include means (such as the processing element 205 of the data object computing entity 105 described above in connection with FIG. 2) to determine a decay indicator.

As described above, the decay indicator is associated with a decrease rate or percentage of a health level associated with a user over a period of time. In some embodiments, the processing element determines a decay indicator based at least in part on the gap indicator determined at step/operation 903 and at least one adjustable indicator. As described above, for each category indicator, the processing element may calculate the decay indicator based at least in part on the following equation:

α_{c}=exp(−Gap_{c}×β+γ) Equation 6

In the above Equation 6, Gap_{c} is the gap indicator associated with a category indicator c, α_{c} is the decay indicator associated with the category indicator c. β and γ are adjustable indicators. In some embodiments, β may be set to 10, and γ may be set to 3. As such, the above equation may be represented as follow:

α_{c}=exp(−Gap_{c}×10+3)

While the description above provides an example calculation of a decay indicator, it is noted that the scope of the present disclosure is not limited to the description above. In some examples, an example decay indicator may be calculated based at least in part on one or more additional and/or alternative algorithms. Additionally, or alternatively, the value of β and/or the value of γ may be set to other values.

In some embodiments, to generate an activity category score indicator associated with a category indicator, the processing element calculates a type denominator indicator for each type indicator under the category indicator and a type numerator indicator for each type indicator under the category indicator.

For example, referring back to FIG. 9, subsequent to and/or in response to step/operation 905, the example method 900 proceeds to step/operation 907. At step/operation 907, a computing entity (such as the data object computing entity 105 described above in connection with FIG. 1 and FIG. 2) may include means (such as the processing element 205 of the data object computing entity 105 described above in connection with FIG. 2) to determine a type denominator indicator.

In some embodiments, the processing element determines the type denominator indicator associated with the type indicator at step/operation 907 based at least in part on a point indicator, a maximum activity indicator associated with the type indicator, and the decay indicator determined at step/operation 905. As described above, the point indicator is associated with the category indicator and the type indicator.

As described above, the maximum activity indicator may refer to a maximum or available number of activities in a particular category and a particular type that a user can participate in or complete. In some embodiments, the adjustable indicator in calculating the decay factor may be based at least in part on or equal to the maximum activity indicator.

As described above, the type denominator indicator describes a goal of activity or activities in a particular category and a particular type that a user should participate in and/or complete. In some embodiments, an example type denominator indicator may be calculated based at least in part on the following algorithm:

$${Denominator}_{c , t} = {\sum }_{i}^{min ( T , N )} {( \frac{1}{i} )}^{{α}_{c}} ⨯ {RP}_{c , t} \qquad Equation 7$$

In the above Equation 7, Denominator_{c,t} is the type denominator indicator associated with a category indicator c and a type indicator t. RP_{c,t} is the point indicator associated with the category indicator c and the type indicator t. α_{c} is the decay indicator associated with the category indicator c calculated at step/operation 905. T is the maximum activity indicator (indicating total number of available activities) associated with a category indicator c and a type indicator t. Because the total number of available activities T may vary by users, the above equation capped the total number of available activities to a value N. In some embodiments, the lesser value between T and N may be 5. As such, the above Equation 7 may be represented as follow:

$${Denominator}_{c , t} = {\sum }_{i}^{5} {( \frac{1}{i} )}^{{α}_{c}} ⨯ {RP}_{c , t} \qquad Equation 8$$

While the description above provides an example calculation of a type denominator indicator, it is noted that the scope of the present disclosure is not limited to the description above. In some examples, an example type denominator indicator may be calculated based at least in part on one or more additional and/or alternative algorithms.

Referring back to FIG. 9, subsequent to and/or in response to step/operation 907, the example method 900 proceeds to step/operation 909. At step/operation 909, a computing entity (such as the data object computing entity 105 described above in connection with FIG. 1 and FIG. 2) may include means (such as the processing element 205 of the data object computing entity 105 described above in connection with FIG. 2) to determine a type numerator indicator.

In some embodiments, the processing element determines the type numerator indicator associated with the type indicator at step/operation 909 based at least in part on a point indicator, an actual activity indicator associated with the type indicator and associated with a client profile data object, and the decay indicator determined at step/operation 905.

As described above, the actual activity indicator reflects a number of activities that the user has done. In some embodiments, the processing element may determine an activity counting time interval indicator (or a “look-back” time window) for determining the number of activities. For example, for activities that recur in low frequency (e.g. once a year), the activity counting time interval indicator or the look-back time window could be longer, such as 12 months so that activities that the user participated in or completed within the past 12 months are reflected in the actual activity indicator. For activities that recur in high frequency (e.g. monthly), the activity counting time interval indicator or the look-back time window could be shorter, such as 3 months so that activities that the user participated in or completed within the past 3 months are reflected in the actual activity indicator. In some embodiments, the adjustable indicator γ in calculating the decay factor may be based at least in part on or equal to the activity counting time interval indicator (or the “look-back” time window).

As described above, the type numerator indicator describes activity or activities in a particular category and a particular type that a user has participated in and/or completed. In some embodiments, an example type numerator indicator may be calculated based at least in part on the following algorithm:

$${Numerator}_{c , t} = min ( {\sum }_{i}^{n} {( \frac{1}{i} )}^{{α}_{c}} ⨯ {RP}_{c , t} , Denominato {r}_{c , t} ) \qquad Equation 9$$

In the above Equation 9, Numerator_{c,t} is the type numerator indicator associated with a category indicator c and a type indicator t. RP_{c,t} is the point indicator associated with the category indicator c and the type indicator t. α_{c} is the decay indicator associated with the category indicator c calculated at step/operation 905. Denominator_{c,t} is the type denominator indicator associated with the category indicator c and the type indicator t calculated at step/operation 907. n is the actual activity indicator associated with the category indicator c and the type indicator t.

While the description above provides an example calculation of a type numerator indicator, it is noted that the scope of the present disclosure is not limited to the description above. In some examples, an example type numerator indicator may be calculated based at least in part on one or more additional and/or alternative algorithms.

Referring back to FIG. 9, subsequent to and/or in response to step/operation 909, the example method 900 proceeds to step/operation 913 and ends.

Optionally, in some embodiments, subsequent to and/or in response to step/operation 909, the example method 900 optionally proceeds to step/operation 911. At step/operation 911, a computing entity (such as the data object computing entity 105 described above in connection with FIG. 1 and FIG. 2) may include means (such as the processing element 205 of the data object computing entity 105 described above in connection with FIG. 2) to generate an activity type score indicator.

In some embodiments, the processing element generates an activity type score indicator based at least in part on the type denominator indicator generated at step/operation 907 and the type numerator indicator generated at step/operation 909. In some embodiments, the activity type score indicator generated at step/operation 911 is associated with the same type indicator and the same category indicator as the type denominator indicator generated at step/operation 907 and the type numerator indicator generated at step/operation 909.

While the description above provides an example calculation of an activity type score indicator, it is noted that the scope of the present disclosure is not limited to the description above. In some examples, an example activity type score indicator may be calculated based at least in part on one or more additional and/or alternative algorithms.

In some embodiments, an example activity type score indicator may be calculated based at least in part on the following algorithm:

$${ActivityTypeScore}_{c , t} = \frac{{Numerator}_{c , t}}{{Denominator}_{c , t}} \qquad Equation 10$$

In the above Equation 10, Denominator_{c,t} is the type denominator indicator associated with a category indicator c and a type indicator t. Numerator_{c,t} is the type numerator indicator associated with the category indicator c and the type indicator t. ActivityTypeScore_{c,t} is the activity type score indicator associated with the category indicator c and the type indicator t.

Referring back to FIG. 9, subsequent to step/operation 911, the example method 900 proceeds to step/operation 913 and ends.

As described above, there are technical challenges, deficiencies and problems associated with data analytics systems, and various example embodiments of the present overcome such challenges. Referring now to FIG. 10, an example method 1000 of generating an example activity category score indicator that overcomes various technical challenges in accordance with embodiments of the present disclosure is illustrated. For example, the example method 1000 generates the example activity category score indicator associated with a category indicator based at least in part on aggregating a plurality of type denominator indicators and a plurality of type numerator indicators associated with a plurality of type indicators under the category indicator.

As shown in FIG. 10, the example method 1000 starts at step/operation 1002.

Subsequent to step/operation 1002, the example method 1000 proceeds to step/operation 1004. At step/operation 1004, a computing entity (such as the data object computing entity 105 described above in connection with FIG. 1 and FIG. 2) may include means (such as the processing element 205 of the data object computing entity 105 described above in connection with FIG. 2) to generate a plurality of type denominator indicators and a plurality of type numerator indicators associated with the category indicator.

For example, for each category indicator, the processing element may determine one or more type indicators under the category indicator, and generate a type denominator indicator and a type numerator indicator for each type indicator. In some embodiments, the processing element may generate a type denominator indicator based at least in part on various examples provided herein, including, but not limited to, those described above in connection with at least step/operation 907 of FIG. 9. In some embodiments, the processing element may generate a type numerator indicator based at least in part on various examples provided herein, including, but not limited to, those described above in connection with at least step/operation 909 of FIG. 9.

For example, TABLE 9 below provides example type indicators associated with the same category indicator, as well as example type numerator indicators and example type denominator indicators:

TABLE 9
EXAMPLE CATEGORY INDICATOR, TYPE
INDICATORS, TYPE NUMERATOR/DENOMINATOR
INDICATORS AND THEIR VALUES

| Category | Type | | Indicator |
|---|---|---|---|
| Indicator | Indicator | Indicator | Value |
| Sleep | Article | Type Numerator Indicator | 277 |
| | | Type Denominator Indicator | 595 |
| | Coaching | Type Numerator Indicator | 310 |
| | | Type Denominator Indicator | 460 |

Referring back to FIG. 10, subsequent to and/or in response to step/operation 1004, the example method 1000 proceeds to step/operation 1006. At step/operation 1006, a computing entity (such as the data object computing entity 105 described above in connection with FIG. 1 and FIG. 2) may include means (such as the processing element 205 of the data object computing entity 105 described above in connection with FIG. 2) to aggregate at least the plurality of type denominator indicators and the plurality of type numerator indicators to generate the activity category score indicator.

As described above, to generate the activity category score indicator, the processing element aggregates indicators from type level to category level. In some embodiments, the processing element may add the type denominator indicators associated with different types indicators under the same category indicator, add the type numerator indicators associated with different types indicators under the same category indicator, and take a fraction.

In some embodiments, an example activity type score indicator may be calculated based at least in part on the following algorithm:

$${ActivityCategoryScore}_{c} = \frac{{\sum }_{t}^{T} {Numerator}_{c , t}}{{\sum }_{t}^{T} {Denominator}_{c , t}} \qquad Equation 11$$

In the above Equation 11, Denominator_{c,t} is the type denominator indicator associated with a category indicator c and a type indicator t. Numerator_{c,t} is the type numerator indicator associated with the category indicator c and the type indicator t. ActivityCategoryScore_{c} is the activity category score associated with the category indicator c. T is the number of type indicator(s) t associated with the category indicator c.

Continuing from the TABLE 9 example above, the example activity category score indicator associated with the sleep category indicator may be calculated as shown below:

$${ActivityCategoryScore}_{sleep} = \frac{227 + 310}{595 + 460} = \frac{537}{1055} \qquad Equation 12$$

Referring back to FIG. 10, subsequent to step/operation 1006, the example method 1000 proceeds to step/operation 1008 and ends.

As described above, there are technical challenges, deficiencies and problems associated with data analytics systems, and various example embodiments of the present overcome such challenges. Referring now to FIG. 11, an example method 1100 of generating an example activity score data object including an activity score indicator that overcomes various technical challenges in accordance with embodiments of the present disclosure is illustrated. For example, the example method 1100 generates the activity score indicator based at least in part on the activity category score indicators associated with a plurality of category indicators and the gap indicators associated with the plurality of category indicators.

As shown in FIG. 11, the example method 1100 starts at step/operation 1101. Subsequent to step/operation 1101, the example method 1100 proceeds to step/operation 1103. At step/operation 1103, a computing entity (such as the data object computing entity 105 described above in connection with FIG. 1 and FIG. 2) may include means (such as the processing element 205 of the data object computing entity 105 described above in connection with FIG. 2) to generate a plurality of activity category score indicators associated with the plurality of category indicators.

In some embodiments, the processing element may generate the plurality of activity category score indicators based at least in part on various examples provided herein, including, but not limited to, those described above in connection with at least FIG. 10. In some embodiments, the plurality of activity category score indicators comprises a plurality of category denominator indicators and a plurality of category numerator indicators.

As described above, the category denominator indicator describes a goal of activity or activities in a particular category that a user should participate in and/or complete. The category numerator indicator describes an accomplishment of activity or activities in a particular category that a user has participated in and/or completed.

In some embodiments, the category denominator indicator of a category indicator may be determined based at least in part on various examples provided herein, including, but not limited to, adding one or more type denominator indicators associated with one or more type indicators under the category indicator, similar to those described above in connection with at least FIG. 10. In some embodiments, the category numerator indicator of a category indicator may be determined based at least in part on various examples provided herein, including, but not limited to, adding one or more type numerator indicators associated with one or more type indicators under the category indicator, similar to those described above in connection with at least FIG. 10.

Referring back to FIG. 11, subsequent to and/or in response to step/operation 1101, the example method 1100 proceeds to step/operation 1105. At step/operation 1105, a computing entity (such as the data object computing entity 105 described above in connection with FIG. 1 and FIG. 2) may include means (such as the processing element 205 of the data object computing entity 105 described above in connection with FIG. 2) to determine a plurality of gap indicators associated with the plurality of category indicators.

In some embodiments, for each category indicator, the processing element calculates a gap indicator. For example, the processing element determines a plurality of gap indicators associated with the plurality of category indicators based at least in part on a plurality of health category score indicators associated with the health score data object, similar to those described above in connection with at least step/operation 903 of FIG. 9.

Referring back to FIG. 11, subsequent to and/or in response to step/operation 1103 and/or step/operation 1105, the example method 1100 proceeds to step/operation 1107. At step/operation 1105, a computing entity (such as the data object computing entity 105 described above in connection with FIG. 1 and FIG. 2) may include means (such as the processing element 205 of the data object computing entity 105 described above in connection with FIG. 2) to generate the activity score data object.

In some embodiments, the processing element generates the activity score data object based at least in part on the plurality of category denominator indicators and the plurality of category numerator indicators generated at step/operation 1103, as well as the plurality of gap indicators generated at step/operation 1105.

For example, the processing element aggregates an activity category score indicator for each category indicator into an activity score indicator for the activity score data object. In some embodiments, an example activity score indicator may be calculated based at least in part on the following algorithm:

$$ActivityScore = \frac{{\sum }_{c}^{C} Numerato {r}_{c} ⨯ ( {Gap}_{c} + ϵ )}{{\sum }_{c}^{C} D enominato {r}_{c} ⨯ ( {Gap}_{c} + ϵ )} \qquad Equation 13$$

In the above Equation 13, ActivityScore is the activity score indicator, Gap_{c} is the gap indicator associated with a category indicator c. Numerator_{c} is the category numerator indicator associated with the category indicator c. Denominator_{c} is the category denominator indicator associated with the category indicator c. C is the total number of category indicators. ε is an adjustable indicator. In some embodiments, the value of ε is set to a small value (such as 0.05) to account for zero gap.

As an example, TABLE 10 below provides example category indicators and their associated health category score indicators, gap indicators, category numerator indicators, and category denominator indicators.

TABLE 10
EXAMPLE CATEGORY INDICATORS, HEALTH
CATEGORY SCORE INDICATORS, GAP INDICATORS,
CATEGORY DENOMINATOR INDICATORS, AND
CATEGORY NUMERATOR INDICATORS

| | | | Category | Category |
|---|---|---|---|---|
| Category | Health Category | Gap | Denominator | Numerator |
| Indicator | Score Indicator | Indicator | Indicator | Indicator |
| Clinical | 0.35 | 0.65 | 100 | 60 |
| Diet | 0.56 | 0.44 | 50 | 40 |
| Exercise | 0.13 | 0.87 | 200 | 20 |
| Sleep | 0.6 | 0.2 | 20 | 10 |

Based at least in part on the example in TABLE 10 and the example equation above, an example activity score indicator may be calculated as follows (where c is set to 0.05):

$$ActivityScore = \frac{60 ⨯ ( 0. 65 + 0.05 ) + 40 ⨯ ( 0. 44 + 0.05 ) + 20 ⨯ ( 0.87 + 0.05 ) + 10 ⨯ ( 0.2 + 0.05 )}{100 ⨯ ( 0. 65 + 0.05 ) + 50 ⨯ ( 0. 44 + 0.05 ) + 200 ⨯ ( 0.87 + 0.05 ) + 20 ⨯ ( 0.2 + 0.05 )}$$

While the description above provides an example calculation of an activity score indicator, it is noted that the scope of the present disclosure is not limited to the description above. In some examples, an example activity score indicator may be calculated based at least in part on one or more additional and/or alternative algorithms.

Referring back to FIG. 11, subsequent to step/operation 1107, the example method 1100 proceeds to step/operation 1109 and ends.

By using the above-described techniques, various embodiments of the present invention introduce techniques for generating activity scores that are configured to be used to recommend activities to an end-user of a device. For example, various embodiments of the present invention introduce techniques for generating activity scores based on point indicators associated with an activity, where each point indicator may be associated with a combination of an activity category indicator and an activity type indicator. By utilizing the noted techniques, various embodiments of the present invention enable techniques for expediated and relatively non-resource-intensive generation of activity scores that enable generating such scores on end-user client devices, rather than on server systems. This in turn reduces the need for client-server communications in order to generate activity scores, which in turn improves network efficiency and network reliability of distributed user activity monitoring systems.

### f. Exemplary Techniques for Updating an Activity Score Data Object and a Composite Score Data Object

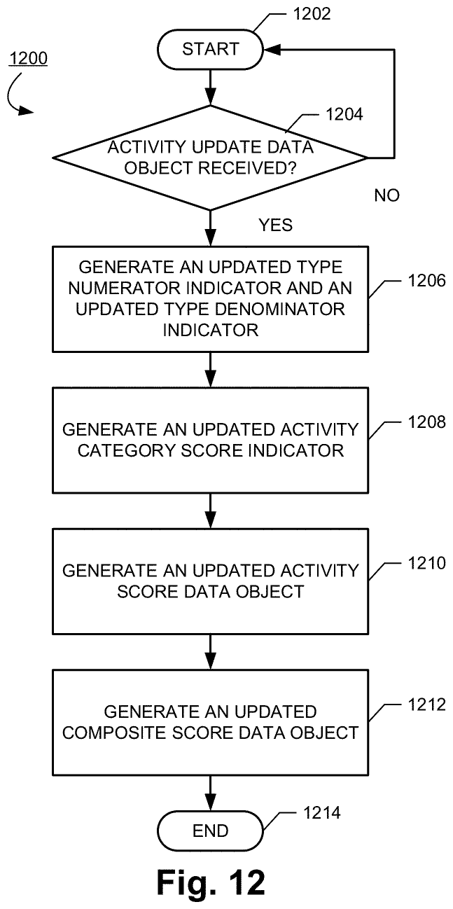

As described above, there are technical challenges, deficiencies and problems associated with data analytic systems, and various example embodiments of the present overcome such challenges. Referring now to FIG. 12, an example method 1200 of generating an updated composite score data object that overcomes various technical challenges in accordance with embodiments of the present disclosure is illustrated. For example, the example method 1200 generates an updated composite score data object based at least in part on an activity update data object.

As shown in FIG. 12, the example method 1200 starts at step/operation 1202. Subsequent to step/operation 1202, the example method 1200 proceeds to step/operation 1204. At step/operation 1204, a computing entity (such as the data object computing entity 105 described above in connection with FIG. 1 and FIG. 2) may include means (such as the processing element 205 of the data object computing entity 105 described above in connection with FIG. 2) to determine whether an activity update data object has been received.

In some embodiments, the processing element may receive an activity update data object associated with a client identifier indicator. As described above, an activity update data object provides data and/or information with regards to updates on additional activities that a user (e.g. associated with the client identifier indicator) has participated in and/or completed.

In some embodiments, an activity update data object may be triggered or generated by a client computing entity, such as, but not limited to, a wearable computer (such as, but not limited to, fitness trackers, smart watches, and/or the like) and/or a mobile computer (such as, but not limited to, smartphones, tablet computers, and/or the like) that monitor and/or track activity related information associated with a user. For example, a user may wear a fitness tracker or carry a smartphone for tracking exercises that the user has participated in or completed. When a user completed an exercise coaching program, the fitness tracker or the smartphone may generate an activity update data object indicating that the user has completed the exercise coaching program.

In some embodiments, the activity update data object comprises an updated actual activity indicator associated with a category indicator and a type indicator. Continuing from the example above, the exercise coaching program is associated with an exercise category indicator and a coaching type indicator. In this example, the fitness tracker or the smartphone may generate an activity update data object comprising an updated actual activity indicator that counts in the completed the exercise coaching program associated with the exercise category indicator and the coaching type indicator.

If, at step/operation 1204, the processing element determines that the activity update data object has not been received, the example method 1200 returns to step/operation 1202. In such an example, the processing element continues to wait until receiving an activity update data object.

If, at step/operation 1204, the processing element determines that the activity update data object has been received, the example method 1200 proceeds to step/operation 1206, step/operation 1208, step/operation 1210, and/or step/operation 1212.

For example, in response to receiving the activity update data object, the processing element generates an updated type numerator indicator and an updated type denominator indicator associated with the type indicator based at least in part on the updated actual activity indicator, generates an updated activity category score indicator associated with the category indicator based at least in part on the updated type numerator indicator and the updated type denominator indicator, generates an updated activity score data object based at least in part on the updated activity category score indicator, and/or generates an updated composite score data object based at least in part on the updated activity score data object, details of which are described herein.

Referring back to FIG. 12, At step/operation 1206, a computing entity (such as the data object computing entity 105 described above in connection with FIG. 1 and FIG. 2) may include means (such as the processing element 205 of the data object computing entity 105 described above in connection with FIG. 2) to generate an updated type numerator indicator and an updated type denominator indicator.

In some embodiments, in response to receiving the activity update data object, the processing element generates an updated type numerator indicator and an updated type denominator indicator associated with the type indicator based at least in part on the updated actual activity indicator associated with the activity update object received at step/operation 1204.

For example, the processing element may generate an updated type numerator indicator based at least in part on various examples provided herein, including, but not limited to, those described above in connection with at least step/operation 909 of FIG. 9. The processing element may generate an updated type denominator indicator based at least in part on various examples provided herein, including, but not limited to, those described above in connection with at least step/operation 907 of FIG. 9.

Referring back to FIG. 12, subsequent to and/or in response to step/operation 1206, the example method 1200 proceeds to step/operation 1208. At step/operation 1208, a computing entity (such as the data object computing entity 105 described above in connection with FIG. 1 and FIG. 2) may include means (such as the processing element 205 of the data object computing entity 105 described above in connection with FIG. 2) to generate an updated activity category score indicator.

In some embodiments, in response to receiving the activity update data object, the processing element generates an updated activity category score indicator associated with the category indicator based at least in part on the updated type numerator indicator and the updated type denominator indicator generated at step/operation 1206.

In some embodiments, the processing element may generate an updated activity category score indicator based at least in part on various examples provided herein, including, but not limited to, those described above in connection with at least FIG. 10.

Referring back to FIG. 12, subsequent to and/or in response to step/operation 1208, the example method 1200 proceeds to step/operation 1210. At step/operation 1210, a computing entity (such as the data object computing entity 105 described above in connection with FIG. 1 and FIG. 2) may include means (such as the processing element 205 of the data object computing entity 105 described above in connection with FIG. 2) to generate an updated activity score data object.

In some embodiments, in response to receiving the activity update data object, the processing element generates an updated activity score data object based at least in part on the updated activity category score indicator generated at step/operation 1208.

In some embodiments, the processing element may generate an updated activity score data object based at least in part on various examples provided herein, including, but not limited to, those described above in connection with at least FIG. 4 and FIG. 11.

Referring back to FIG. 12, subsequent to and/or in response to step/operation 1210, the example method 1200 proceeds to step/operation 1212. At step/operation 1212, a computing entity (such as the data object computing entity 105 described above in connection with FIG. 1 and FIG. 2) may include means (such as the processing element 205 of the data object computing entity 105 described above in connection with FIG. 2) to generate an updated composite score data object.

In some embodiments, in response to receiving the activity update data object, the processing element generates an updated composite score data object based at least in part on the updated activity score data object generated at step/operation 1210.

In some embodiments, the processing element may generate an updated composite score data object based at least in part on various examples provided herein, including, but not limited to, those described above in connection with at least FIG. 4 to FIG. 13B.

Referring back to FIG. 12, subsequent to step/operation 1212, the example method 1200 proceeds to step/operation 1214 and ends.

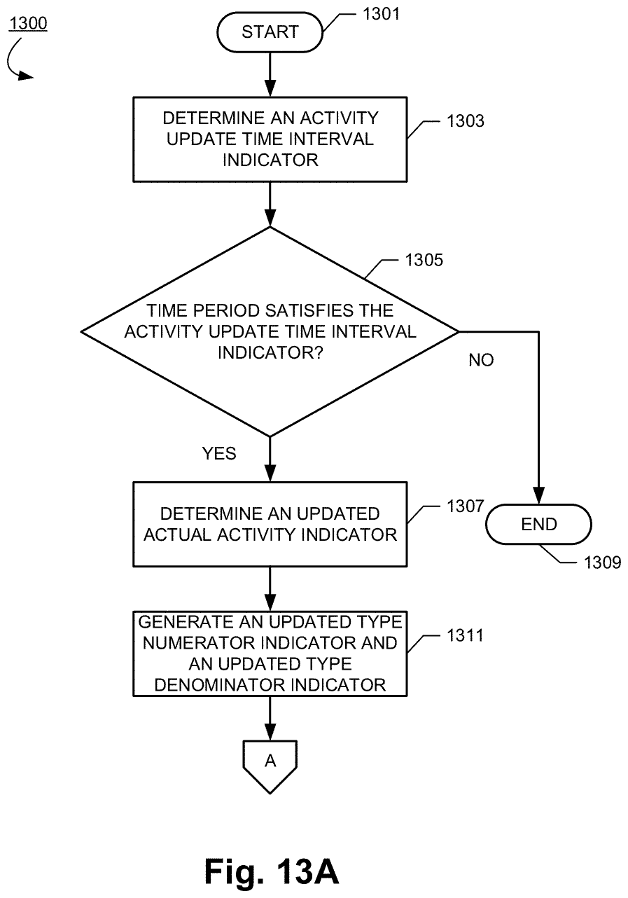

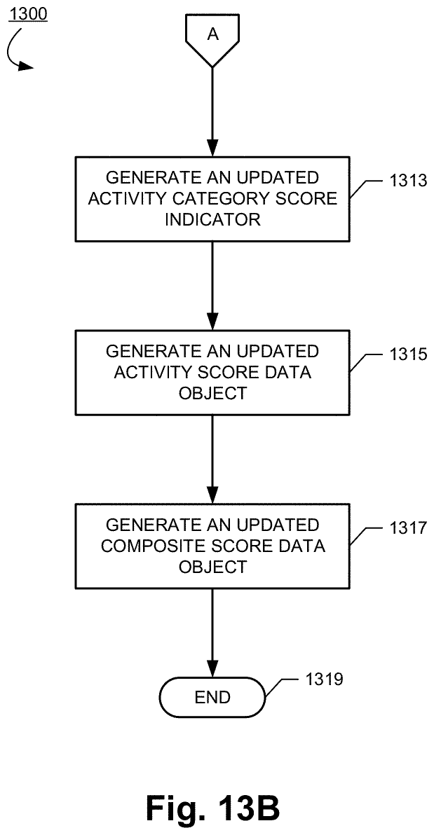

As described above, there are technical challenges, deficiencies and problems associated with data analytic systems, and various example embodiments of the present overcome such challenges. For example, referring now to FIG. 13A and FIG. 13B, an example method 1300 of generating an updated composite score data object that overcomes various technical challenges in accordance with embodiments of the present disclosure is illustrated. For example, the example method 1300 generates an updated composite score data object based at least in part on determining whether a time period between the composite score data object creation time indicator and a current time indicator satisfies an activity update time interval indicator.

As shown in FIG. 13A, the example method 1300 starts at step/operation 1301. Subsequent to step/operation 1301, the example method 1300 proceeds to step/operation 1303. At step/operation 1303, a computing entity (such as the data object computing entity 105 described above in connection with FIG. 1 and FIG. 2) may include means (such as the processing element 205 of the data object computing entity 105 described above in connection with FIG. 2) to determine an activity update time interval indicator.

As described above, an activity update time interval indicator may refer to a time window for updating the activity score data object or the composite score data object. In some embodiments, the activity update time interval indicator may be set by the example activity score data object and/or composite score data object generating platform/system. In some embodiments, the activity update time interval indicator may be set by a user.

Referring back to FIG. 13A, subsequent to and/or in response to step/operation 1303, the example method 1300 proceeds to step/operation 1305. At step/operation 1305, a computing entity (such as the data object computing entity 105 described above in connection with FIG. 1 and FIG. 2) may include means (such as the processing element 205 of the data object computing entity 105 described above in connection with FIG. 2) determines whether a time period satisfies the activity update time interval indicator.

In some embodiments, the processing element determines whether a time period between a composite score data object creation time indicator associated with a user and a current time indicator satisfies the activity update time interval indicator. For example, if the time period between the composite score data object creation time indicator and the current time indicator is 12 hours, and the activity update time interval indicator is 24 hours, the processing element determines that the time period is less than the activity update time interval indicator and therefore does not satisfy the activity update time interval indicator. If the time period between the composite score data object creation time indicator and the current time indicator is 36 hours, and the activity update time interval indicator is 24 hours, the processing element determines that the time period is not less than (e.g. equal to or more than) the activity update time interval indicator and therefore satisfies the activity update time interval indicator.

In some embodiments, the processing element determines whether a time period between the activity score data object creation time indicator associated with a user and a current time indicator satisfies the activity update time interval indicator. For example, if the time period between the activity score data object creation time indicator and the current time indicator is 12 hours, and the activity update time interval indicator is 24 hours, the processing element determines that the time period is less than the activity update time interval indicator and therefore does not satisfy the activity update time interval indicator. If the time period between the activity score data object creation time indicator and the current time indicator is 36 hours, and the activity update time interval indicator is 24 hours, the processing element determines that the time period is not less than (e.g. equal to or more than) the activity update time interval indicator and therefore satisfies the activity update time interval indicator.

Referring back to FIG. 13A, if, at step/operation 1305, the processing element determines that the time period does not satisfy the activity update time interval indicator, the example method 1300 proceeds to step/operation 1309 and ends.

If, at step/operation 1305, the processing element determines that the time period does satisfy the activity update time interval indicator, the example method 1300 proceeds to step/operation 1307, step/operation 1311, step/operation 1313, step/operation 1315, and/or step/operation 1317.

For example, in response to determining that the time period satisfies the activity update time interval indicator, the processing element determines an updated actual activity indicator associated with at least one of the category indicator or the type indicator, generates an updated type numerator indicator and an updated type denominator indicator associated with the type indicator based at least in part on the updated actual activity indicator, generates an updated activity category score indicator associated with the category indicator based at least in part on the updated type numerator indicator and the updated type denominator indicator, generates an updated activity score data object based at least in part on the updated activity category score indicator, and/or generates an updated composite score data object based at least in part on the updated activity score data object.

Referring back to FIG. 13A, at step/operation 1307, a computing entity (such as the data object computing entity 105 described above in connection with FIG. 1 and FIG. 2) may include means (such as the processing element 205 of the data object computing entity 105 described above in connection with FIG. 2) to determine an updated actual activity indicator.

In some embodiments, the processing element retrieves a client activity data object from a database, similar to those described above in connection with at least FIG. 4. In some embodiments, the client activity data object provides an updated actual activity indicator that is associated with at least one of the category indicator or the type indicator. For example, the client activity data object may indicate that a user has completed an exercise coaching program subsequent to the last time that an activity data object and/or a composite data object was generated.

Referring back to FIG. 13A, subsequent to and/or in response to step/operation 1307, the example method 1300 proceeds to step/operation 1311. At step/operation 1311, a computing entity (such as the data object computing entity 105 described above in connection with FIG. 1 and FIG. 2) may include means (such as the processing element 205 of the data object computing entity 105 described above in connection with FIG. 2) to generate an updated type numerator indicator and an updated type denominator indicator.

In some embodiments, the processing element generates the updated type numerator indicator and the updated type denominator indicator associated with the type indicator based at least in part on the updated actual activity indicator determined at step/operation 1307.

For example, in response to determining that the time period satisfies the activity update time interval indicator, the processing element may generate an updated type numerator indicator based at least in part on various examples provided herein, including, but not limited to, those described above in connection with at least step/operation 909 of FIG. 9. Additionally, in response to determining that the time period satisfies the activity update time interval indicator, the processing element may generate an updated type denominator indicator based at least in part on various examples provided herein, including, but not limited to, those described above in connection with at least step/operation 907 of FIG. 9.

Referring back to FIG. 13A, subsequent to and/or in response to step/operation 1311, the example method 1300 proceeds to block A, which connects FIG. 13A to FIG. 13B. As shown in FIG. 13B, subsequent to and/or in response to step/operation 1311, the example method 1300 proceeds to step/operation 1313, a computing entity (such as the data object computing entity 105 described above in connection with FIG. 1 and FIG. 2) may include means (such as the processing element 205 of the data object computing entity 105 described above in connection with FIG. 2) to generate an updated activity category score indicator.

In some embodiments, in response to determining that the time period satisfies the activity update time interval indicator, the processing element generates an updated activity category score indicator associated with the category indicator based at least in part on the updated type numerator indicator and the updated type denominator indicator generated at step/operation 1311.

In some embodiments, the processing element may generate an updated activity category score indicator based at least in part on various examples provided herein, including, but not limited to, those described above in connection with at least FIG. 10.

Referring back to FIG. 13B, subsequent to and/or in response to step/operation 1313, the example method 1300 proceeds to step/operation 1315. At step/operation 1315, a computing entity (such as the data object computing entity 105 described above in connection with FIG. 1 and FIG. 2) may include means (such as the processing element 205 of the data object computing entity 105 described above in connection with FIG. 2) to generate an updated activity score data object.

In some embodiments, in response to determining that the time period satisfies the activity update time interval indicator, the processing element generates an updated activity score data object based at least in part on the updated activity category score indicator generated at step/operation 1313.

In some embodiments, the processing element may generate an updated activity score data object based at least in part on various examples provided herein, including, but not limited to, those described above in connection with at least FIG. 4 and FIG. 11.

Referring back to FIG. 13B, subsequent to and/or in response to step/operation 1315, the example method 1300 proceeds to step/operation 1317. At step/operation 1317, a computing entity (such as the data object computing entity 105 described above in connection with FIG. 1 and FIG. 2) may include means (such as the processing element 205 of the data object computing entity 105 described above in connection with FIG. 2) to generate an updated composite score data object.

In some embodiments, in response to determining that the time period satisfies the activity update time interval indicator, the processing element generates an updated composite score data object based at least in part on the updated activity score data object generated at step/operation 1315.

In some embodiments, the processing element may generate an updated composite score data object based at least in part on various examples provided herein, including, but not limited to, those described above in connection with at least FIG. 4 to FIG. 12.

Referring back to FIG. 13B, subsequent to step/operation 1317, the example method 1300 proceeds to step/operation 1319 and ends.

Thus, as described, various embodiments of the present invention introduce techniques for generating activity scores that are configured to be used to recommend activities to an end-user of a device. For example, various embodiments of the present invention introduce techniques for generating activity scores based on point indicators associated with an activity, where each point indicator may be associated with a combination of an activity category indicator and an activity type indicator. By utilizing the noted techniques, various embodiments of the present invention enable techniques for expediated and relatively non-resource-intensive generation of activity scores that enable generating such scores on end-user client devices, rather than on server systems. This in turn reduces the need for client-server communications in order to generate activity scores, which in turn improves network efficiency and network reliability of distributed user activity monitoring systems.

### g. Exemplary Techniques for Performing Score-Based Actions Based at Least in Part on Composite Score Data Object

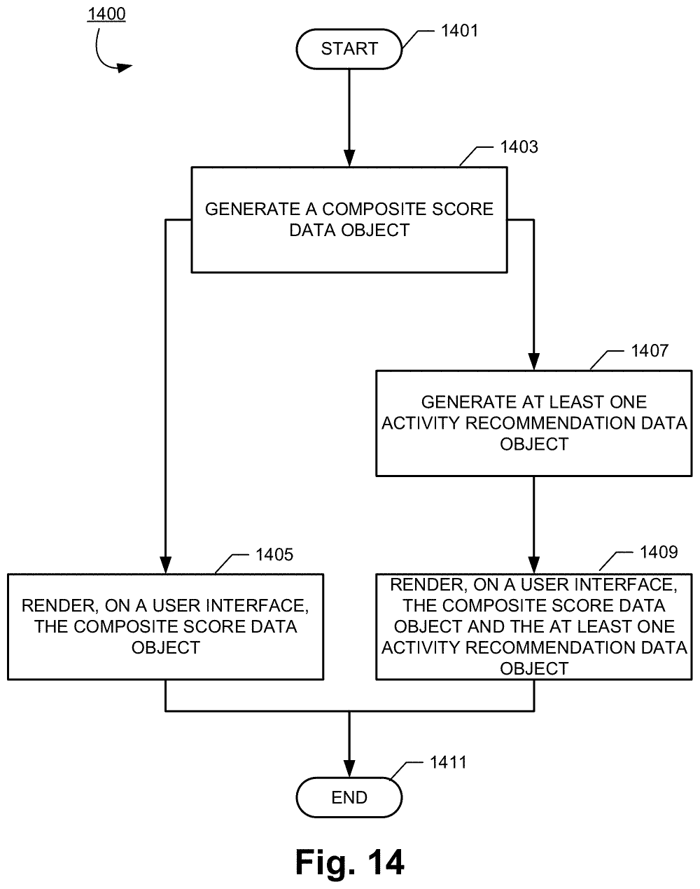

As described above, there are technical challenges, deficiencies and problems associated with data analytic systems, and various example embodiments of the present overcome such challenges. Referring now to FIG. 14, an example method 1400 of conducting score-based actions based at least in part on the composite score data object that overcomes various technical challenges in accordance with embodiments of the present disclosure is illustrated. For example, the example method 1400 may render the composite score data object and/or activity recommendation data objects generated based at least in part on the composite score data object on a user interface.

As shown in FIG. 14, the example method 1400 starts at step/operation 1401. Subsequent to step/operation 1401, the example method 1400 proceeds to step/operation 1403. At step/operation 1403, a computing entity (such as the data object computing entity 105 described above in connection with FIG. 1 and FIG. 2) may include means (such as the processing element 205 of the data object computing entity 105 described above in connection with FIG. 2) to generate a composite score data object.

In some embodiments, the processing element may generate a composite score data object based at least in part on various examples provided herein, including, but not limited to, those described above in connection with at least FIG. 4 to FIG. 13B.

Referring back to FIG. 14, subsequent to and/or in response to step/operation 1403, the example method 1400 proceeds to step/operation 1405. At step/operation 1405, a computing entity (such as the data object computing entity 105 described above in connection with FIG. 1 and FIG. 2) may include means (such as the processing element 205 of the data object computing entity 105 described above in connection with FIG. 2) to render, on a user interface, the composite score data object.

In some embodiments, the processing element renders the composite score data object on a user interface displayed on a client computing entity associated with the client identifier indicator. For example, the processing element may render the composite score indicator of the composite score data object on a user interface displayed on a wearable computer (such as, but not limited to, fitness trackers, smart watches, and/or the like) and/or a mobile computer (such as, but not limited to, smartphones, tablet computers, and/or the like). An example user interface is illustrated and described in connection with at least FIG. 16.

Referring back to FIG. 14, subsequent to step/operation 1405, the example method 1400 proceeds to step/operation 1411 and ends.

Referring back to FIG. 14, subsequent to and/or in response to step/operation 1403, the example method 1400 proceeds to step/operation 1407. At step/operation 1407, a computing entity (such as the data object computing entity 105 described above in connection with FIG. 1 and FIG. 2) may include means (such as the processing element 205 of the data object computing entity 105 described above in connection with FIG. 2) to generate at least one activity recommendation data object.

In some embodiments, the processing element generates at least one activity recommendation data object based at least in part on the composite score data object and the client profile data object. For example, the client profile data object is associated with a client preference indicator, and the processing element generates at least one activity recommendation data object based further on the client preference indicator.

As described above, the client preference indicator describes one or more preferences associated with a user (e.g. associated with a client identifier indicator), including, but not limited to, activities that the user likes to participate, activities that the user does not like to participate, a priority of activities for the user, and/or the like. In some example, based at least in part on the composite score indicator of the composite score data object being lower than a threshold, the processing element may generate at least one activity recommendation data object that recommends an activity to a user to improve the composite score indicator, and the activity is one of the activities that the user likes to participate based at least in part on the client preference indicator.

As described above, the composite score data object is generated based at least in part on an activity score data object, which in turn is generated based at least in part on activity category score indicators associated with a plurality of category indicators. In some embodiments, based at least in part on an activity category score indicator associated with a category indicator being lower than a threshold, the processing element may generate at least one activity recommendation data object that recommends an activity associated with that category indicator.

Referring back to FIG. 14, subsequent to and/or in response to step/operation 1407, the example method 1400 proceeds to step/operation 1409. At step/operation 1409, a computing entity (such as the data object computing entity 105 described above in connection with FIG. 1 and FIG. 2) may include means (such as the processing element 205 of the data object computing entity 105 described above in connection with FIG. 2) to render, on a user interface, the composite score data object and the at least one activity recommendation data object.

In some embodiments, the processing element renders the composite score data object and the at least one activity recommendation data object on a user interface displayed on a client computing entity associated with the client identifier indicator, similar to those described in connection with at least step/operation 1405.

Referring back to FIG. 14, subsequent to step/operation 1409, the example method 1400 proceeds to step/operation 1411 and ends.

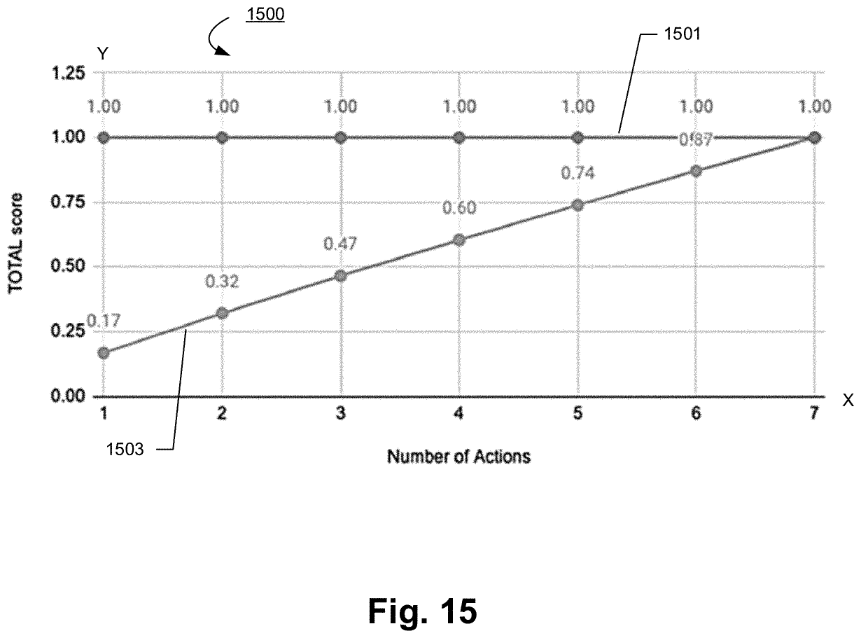

Referring now to FIG. 15, an example diagram 1500 is illustrated. In particular, the example diagram 1500 illustrates example data colorations between an health score data object and an activity score data object associated with a user. In particular, the example diagram 1500 illustrates that an activity score indicator not only depends on the number of actions that a user takes, but also depends on the user's health.

In diagram 1500, the curve 1501 is associated with a user having a health score indicator of 1, and the curve 1503 is associated with a user with a health score indicator of 0.5. The x axis indicates a total number of actions that a user takes (e.g. an actual action indicator associated with a category indicator and a type indicator), and the y axis indicates the corresponding activity score indicator. The activity score indicators shown in FIG. 15 may be calculated in accordance with various examples described herein, including, but not limited to, those described in connection with at least FIG. 4 to FIG. 13B.

As shown in FIG. 15, a healthy user (as reflected in curve 1501) only needs to take 1 action to get a full activity score indicator of 1. For less and less healthy users (as reflected in curve 1503), they need to keep taking more and more actions to get a full activity score. As such, a healthy user can achieve the goal and earn full points by taking few actions. In contract, a less healthy user has to take more actions to achieve the “goal” and earn full points. In some embodiments, in categories that a user is underperforming, the corresponding activity category score indicator may have more weights on the users' composite score indicator.

As such, various embodiments of the present disclosure personalize the activity score data object based at least in part on the health level associated with the user, and encourages less healthy users to participate in more healthy activities.

Referring now to FIG. 16, an example user interface 1600 in accordance with various embodiments of the present disclosure is illustrated.

In the example shown in FIG. 16, the example user interface 1600 comprises a composite score section 1603, which comprises a rendering of a composite score data object that indicates a current composite score indicator associated with the user. The example user interface 1600 comprises a trend section 1601, which illustrates a historical trend associated with historical composite score indicators of the user.

In some embodiments, the example user interface 1600 comprises a plurality of category icons, each associated with a corresponding category indicator. For example, the example user interface 1600 illustrates an example category icon 1605A associated with a sleep category indicator, an example category icon 1605B associated with an exercise category indicator, an example category icon 1605C associated with a nutrition category indicator, an example category icon 1605D associated with a mood category indicator, an example category icon 1605E associated with a smoking category indicator, an example category icon 1605F associated with an alcohol category indicator, an example category icon 1605G associated with a conditions category indicator. When a user selects a category icon, the example user interface 1600 is updated to display data and/or information associated with the corresponding category indicator.

For example, as shown in FIG. 16, the user clicks the category icon 1605A associated with a sleep category indicator, and the user interface is updated to display data and/or information associated with the sleep category. For example, the example user interface 1600 comprises an activity category score section 1607, which comprises a rendering of an activity category score data object that indicates a current activity category score indicator associated with the user and the category indicator (e.g. sleep). The example user interface 1600 comprises a regeneration button 1609, which is configured to trigger generating an updated activity score data object when selected based at least in part on various examples described herein.

The example user interface 1600 comprises an activity overview section 1611, which graphically illustrates an overview of the number of activities that a user has participated in or completed under the category indicator. The example user interface 1600 comprises an explanation section 1613, which comprises texts that explain how the activity category score indicator shown in activity category score section 1607 is generated. The example user interface 1600 comprises an activity recommendation section 1605, which may textually and/or graphically illustrate one or more recommended activities (for example, missions, programs, challenges, and/or articles) associated with the category indicator for the user to participate in or complete in order to improve the activity category score indicator.

While the description above provides an example of user interface, it is noted that the scope of the present disclosure is not limited to the description above. In some examples, an example user interface may comprise one or more additional and/or alternative elements.

### V. CONCLUSION

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. An apparatus comprising at least one processor and at least one non-transitory memory comprising a computer program code, the at least one non-transitory memory and the computer program code configured to, with the at least one processor, cause the apparatus to:
retrieve a client profile data object based at least in part on a client identifier indicator, wherein the client profile data object is associated with a health score data object and at least one client activity data object;
determine a plurality of point indicators based at least in part on the at least one client activity data object, wherein the plurality of point indicators are associated with a plurality of category indicators and a plurality of type indicators;
generate, based at least in part on the plurality of category indicators, the plurality of type indicators, the plurality of point indicators, and the health score data object, an activity score data object associated with the client profile data object;
generate a composite score data object based at least in part on the health score data object and the activity score data object, wherein the composite score data object is associated with the client profile data object; and
perform at least one score-based action based at least in part on the composite score data object and the client profile data object.

2. The apparatus of claim 1, wherein, when determining the plurality of point indicators, the at least one non-transitory memory and the computer program code are configured to, with the at least one processor, cause the apparatus to:
determine a point indicator of the plurality of point indicators based at least in part on a category indicator of the plurality of category indicators, wherein the point indicator is associated with the category indicator.

3. The apparatus of claim 1, wherein, when determining the plurality of point indicators, the at least one non-transitory memory and the computer program code are configured to, with the at least one processor, cause the apparatus to:
determine a point indicator of the plurality of point indicators based at least in part on a type indicator of the plurality of type indicators, wherein the point indicator is associated with the type indicator.

4. The apparatus of claim 1, wherein, when determining the plurality of point indicators, the at least one non-transitory memory and the computer program code are configured to, with the at least one processor, cause the apparatus to:
determine a point indicator of the plurality of point indicators based at least in part on a category indicator of the plurality of category indicators and a type indicator of the plurality of type indicators, wherein the point indicator is associated with the category indicator and the type indicator.

5. The apparatus of claim 1, wherein the health score data object comprises a health category score indicator, wherein the health category score indicator is associated with a category indicator of the plurality of category indicators.

6. The apparatus of claim 5, wherein, when generating the activity score data object, the at least one non-transitory memory and the computer program code are configured to, with the at least one processor, cause the apparatus to:
generate an activity category score indicator associated with the category indicator based at least in part on the health category score indicator.

7. The apparatus of claim 6, wherein, when generating the activity category score indicator, the at least one non-transitory memory and the computer program code are configured to, with the at least one processor, cause the apparatus to:
determine a gap indicator based at least in part on the health category score indicator and a health category maximum indicator associated with the category indicator; and
determine a decay indicator based at least in part on the gap indicator and at least one adjustable indicator.

8. The apparatus of claim 7, wherein a point indicator of the plurality of point indicators is associated with the category indicator and a type indicator of the plurality of type indicators, wherein the at least one non-transitory memory and the computer program code are configured to, with the at least one processor, cause the apparatus to:
determine a type denominator indicator associated with the type indicator based at least in part on the point indicator, a maximum activity indicator associated with the type indicator, and the decay indicator; and
determine a type numerator indicator associated with the type indicator based at least in part on the point indicator, an actual activity indicator associated with the type indicator and the client profile data object, and the decay indicator.

9. The apparatus of claim 8, wherein the at least one non-transitory memory and the computer program code are configured to, with the at least one processor, cause the apparatus to:
generate an activity type score indicator based at least in part on the type denominator indicator and the type numerator indicator, wherein the activity type score indicator is associated with the type indicator and the category indicator.

10. The apparatus of claim 6, wherein, when generating the activity category score indicator, the at least one non-transitory memory and the computer program code are configured to, with the at least one processor, cause the apparatus to:
generate a plurality of type denominator indicators and a plurality of type numerator indicators associated with the category indicator; and
aggregate at least the plurality of type denominator indicators and the plurality of type numerator indicators to generate the activity category score indicator.

11. The apparatus of claim 1, wherein, when generating the composite score data object, the at least one non-transitory memory and the computer program code are configured to, with the at least one processor, cause the apparatus to:
generate a plurality of activity category score indicators associated with the plurality of category indicators, wherein the plurality of activity category score indicators comprises a plurality of category denominator indicators and a plurality of category numerator indicators;
determine a plurality of gap indicators associated with the plurality of category indicators based at least in part on a plurality of health category score indicators associated with the health score data object; and
generate the activity score data object based at least in part on the plurality of category denominator indicators, the plurality of category numerator indicators, and the plurality of gap indicators.

12. The apparatus of claim 1, wherein the at least one non-transitory memory and the computer program code are configured to, with the at least one processor, cause the apparatus to:
receive an activity update data object associated with the client identifier indicator, wherein the activity update data object comprises an updated actual activity indicator associated with a category indicator and a type indicator;
in response to receiving the activity update data object:
generate an updated type numerator indicator and an updated type denominator indicator associated with the type indicator based at least in part on the updated actual activity indicator;
generate an updated activity category score indicator associated with the category indicator based at least in part on the updated type numerator indicator and the updated type denominator indicator;
generate an updated activity score data object based at least in part on the updated activity category score indicator; and
generate an updated composite score data object based at least in part on the updated activity score data object.

13. The apparatus of claim 1, wherein the composite score data object is associated with a composite score data object creation time indicator, wherein the at least one non-transitory memory and the computer program code are configured to, with the at least one processor, cause the apparatus to:
determine an activity update time interval indicator;
determine whether a time period between the composite score data object creation time indicator and a current time indicator satisfies the activity update time interval indicator; and
in response to determining that the time period satisfies the activity update time interval indicator:
determine an updated actual activity indicator associated with at least one of a category indicator or a type indicator;
generate an updated type numerator indicator and an updated type denominator indicator associated with the type indicator based at least in part on the updated actual activity indicator;
generate an updated activity category score indicator associated with the category indicator based at least in part on the updated type numerator indicator and the updated type denominator indicator;
generate an updated activity score data object based at least in part on the updated activity category score indicator; and
generate an updated composite score data object based at least in part on the updated activity score data object.

14. The apparatus of claim 1, wherein, when performing the at least one score-based action, the at least one non-transitory memory and the computer program code are configured to, with the at least one processor, cause the apparatus to:
render, on a user interface displayed on a client computing entity associated with the client identifier indicator, the composite score data object.

15. The apparatus of claim 1, wherein, when performing the at least one score-based action, the at least one non-transitory memory and the computer program code are configured to, with the at least one processor, cause the apparatus to:
generate at least one activity recommendation data object based at least in part on the composite score data object and the client profile data object; and
render, on a user interface displayed on a client computing entity associated with the client identifier indicator, the at least one activity recommendation data object.

16. The apparatus of claim 15, wherein the client profile data object comprises a client preference indicator, wherein the at least one non-transitory memory and the computer program code are configured to, with the at least one processor, cause the apparatus to:
generate the at least one activity recommendation data object based further on the client preference indicator.

17. A computer implemented method comprising:
retrieving, using a processor, a client profile data object based at least in part on a client identifier indicator, wherein the client profile data object is associated with a health score data object and at least one client activity data object;
determining, using the processor, a plurality of point indicators based at least in part on the at least one client activity data object, wherein the plurality of point indicators are associated with a plurality of category indicators and a plurality of type indicators;
generating, using the processor and based at least in part on the plurality of category indicators, the plurality of type indicators, the plurality of point indicators, and the health score data object, an activity score data object associated with the client profile data object;
generating, using the processor, a composite score data object based at least in part on the health score data object and the activity score data object, wherein the composite score data object is associated with the client profile data object; and
performing, using the processor, at least one score-based action based at least in part on the composite score data object and the client profile data object.

18. The computer implemented method of claim 17, wherein determining the plurality of point indicators further comprises:
determining a point indicator of the plurality of point indicators based at least in part on a category indicator of the plurality of category indicators, wherein the point indicator is associated with the category indicator.

19. The computer implemented method of claim 17, wherein determining the plurality of point indicators further comprises:
determining a point indicator of the plurality of point indicators based at least in part on a type indicator of the plurality of type indicators, wherein the point indicator is associated with the type indicator.

20. A computer program product comprising at least one non-transitory computer-readable storage medium having computer-readable program code portions stored therein, the computer-readable program code portions comprising an executable portion configured to:
retrieve a client profile data object based at least in part on a client identifier indicator, wherein the client profile data object is associated with a health score data object and at least one client activity data object;
determine a plurality of point indicators based at least in part on the at least one client activity data object, wherein the plurality of point indicators are associated with a plurality of category indicators and a plurality of type indicators;
generate, based at least in part on the plurality of category indicators, the plurality of type indicators, the plurality of point indicators, and the health score data object, an activity score data object associated with the client profile data object;
generate a composite score data object based at least in part on the health score data object and the activity score data object, wherein the composite score data object is associated with the client profile data object; and
perform at least one score-based action based at least in part on the composite score data object and the client profile data object.
